(19) United States
(12) Patent Application Publication
(10) Pub. No.: US 2025/0282815 A1
(43) Pub. Date: Sep. 11, 2025

(54) IMPROVED PROTEIN PURIFICATION

(71) Applicant: Cytiva Bioprocess R&D AB, Uppsala (SE)

(72) Inventors: Peter Lundback, Uppsala (SE); Johan Fredrik Ohman, Uppsala (SE)

(21) Appl. No.: 18/555,848

(22) PCT Filed: May 9, 2022
(86) PCT No.: PCT/EP2022/062467

§ 371 Date: Oct. 17, 2023

(30) Foreign Application Priority Data
May 12, 2021 (GB) 2106771.5

(51) Int. Cl. C07K 1/22; C07K 14/195

(52) U.S. Cl. CPC C07K 1/22; C07K 14/195

(57) ABSTRACT

The present invention relates to protein purification, primarily in the chromatographic field. More closely, the invention relates to affinity chromatography using a split intein system comprising a C-intein tag and N-intein ligand, wherein the N-intein ligand provides increased solubility suitable for large scale purification of any recombinant target protein.

### FIELD OF THE INVENTION

The present invention relates to protein purification, primarily in the chromatographic field. More closely, the invention relates to affinity chromatography using a split intein system comprising a C-intein tag and N-intein ligand, wherein the N-intein ligand has high solubility and may be immobilized to a solid phase in high degree suitable for large scale protein purification.

### BACKGROUND OF THE INVENTION

Inteins are protein elements expressed as in-frame insertions that interrupt enzyme sequences and catalyze their own excision and ligation of two flanking polypeptides, generating an active protein. Genetically, inteins are encoded in two distinct ways: as intact inteins, interrupting two flanking extein sequences, or as split inteins, wherein each extein and part of the intein are encoded by two different genes. While they hold great promise as bioengineering and protein purification tools, split inteins with rapid kinetic properties found in nature are dependent on specific amino acids at the intein-extein junction, severely limiting the proteins that can be fused to inteins for affinity purification and recovery of native protein sequences. In particular, the prototypical split intein DNAE from Nostoc punctiforme exhibits kinetic properties suitable for protein purification applications. However, its activity is dependent on phenylalanine at the +2 position in the C-extein. This dependency severely narrows and impairs its general applicability.

Inteins have been engineered to accomplish several important functions in biotechnology, including applications as self-cleaving proteins for recombinant protein purification. Split inteins are particularly promising in this regard, as they can simultaneously provide affinity ligand and self-cleavage properties. In protein purification, a target protein that is the subject of purification may be substituted for either extein. To date, the DNAE family of split inteins has shown the most promise with C-terminal cleavage protein purification approaches.

WO2014/004336 describes proteins fused to split intein N-fragments and split intein C-fragments which could be attached to a support. The solid support could be a particle, bead, resin, or a slide.

WO2014/110393 describes proteins of interest fused to a split intein C-fragment which is contacted with a split intein N-fragment and a purification tag. The N-fragment may be attached to a solid phase via the purification tag and methods for affinity purification are discussed.

U.S. Pat. No. 10,066,027 describes a protein purification system and methods of using the system. Disclosed is a split intein comprising an N-terminal intein segment, which can be immobilized, and a C-terminal intein segment, which has the property of being self-cleaving, and which can be attached to a protein of interest The N-terminal intein segment is provided with a sensitivity enhancing motif which renders it more sensitive to extrinsic conditions.

U.S. Pat. No. 10,308,679 describes fusion proteins comprising an N-intein polypeptide and N-intein solubilization partner, and affinity matrices comprising such fusion proteins.

WO 2018/091424 describes a method for production of an affinity chromatography resin comprising an amino-terminal, (N-terminal), split intein fragment as an affinity ligand, comprising the following steps: a) expression of an N-terminal split intein fragment protein as insoluble protein in inclusion bodies in bacterial cells, preferably E. coli, b) harvesting said inclusion bodies; c) solubilizing said inclusion bodies and releasing expressed protein; d) binding said protein on a solid support; e) refolding said protein; f) releasing said protein from the solid support; and g) immobilizing said protein as ligands on a chromatography resin to form an affinity chromatography resin. This procedure enables immobilization a ligand density of 2-10 mg/ml resin.

As described above, split inteins have been used for protein purification using a combined affinity tag and tag cleavage mechanism. However, the utility of such systems, is limited by several factors. First, there is the amino acid requirements at the splice junction of the intended product, i.e. the requirement of Phe in the +2 position of the C-extein, to effect cleavage and attain purification of tag-less proteins. Recombinant protein production without extraneous amino acid on the N-terminus is highly desirable. Second, the protein releasing cleavage has to be sufficiently fast and provide an acceptable yield. Third, there is a solubility requirement of the split intein N- or C-fragment for attachment thereof to a solid support. Fourth, hitherto there are no available split intein systems suitable for large scale purification of tag-less proteins.

One way to improve the solubility is by attaching a solubility fusion-tag to the split-inteins, (U.S. Pat. No. 10,308,679). The development of methods for protein expression and purification is commonly facilitated by the use of fusion tags that offer the possibility to standardize protocols for purification, simplify the detection and increase the solubility of a target protein. Fusion tags can however interfere with protein function and structure. It is therefore advantageous to remove fusion tags prior to usage. A large fusion tag relative to a target protein also results in an increased metabolic burden for the host cells expressing these fusion proteins, since additional energy is spent on the fusion tag.

A different approach to increase the efficiency of producing highly insoluble split-inteins is by solubilizing proteins with denaturing chemical reagents followed by a refolding process, (U.S. application Ser. No. 16/348,534) to regain bioactive protein. Attempts have been made to understand the technical aspects of various methods used for protein refolding along with their advantages and limitations, but usually the efficiency and yield in such methods is very difficult to predict and has to be determined by empirical studies for each protein. A common problem in refolding methods is the formation of protein aggregates when the denaturing chemicals are being removed or diluted during refolding. These aggregates lower the yield in the process and adds complexity during the subsequent purification steps in a production process. Moreover, the denaturing chemicals are usually a burden to the environment and needs to be properly handled.

### SUMMARY OF THE INVENTION

The present invention overcomes the disadvantages within prior art and provides a N-intein polypeptide, which is soluble without the need for a solubility fusion-tag and that can be produced in an industrial scale in an environmentally friendly production process for subsequent use in affinity purification processes.

In particular, the invention provides a method to increase the solubility of the prototypical split intein DnaE from Nostoc punctiforme, which exhibits kinetic properties suitable for protein purification applications. However, the method is not limited to DnaE split intein from Nostoc punctiforme but is also applicable for homologous split inteins from other species. Solubility refers to a protein that after a substitution of one or preferably two amino acids in the polypeptide chain has a higher ratio of soluble N-intein expressed in E. coli relative to the soluble N-intein ratio in the absence of these amino acid substitutions.

The present invention provides N-intein protein variants of native split inteins or consensus sequences derived from inteins/split inteins wherein the N-intein protein variant has one or more mutations for increased solubility.

In a first aspect, the invention relates to an N-intein variant derived from native Nostoc punctiforme (Npu) or sequences having at least 95% homology therewith comprising at least one amino acid substitution of a native split intein wherein the N-intein protein variant sequence includes a mutation in at least position 24 and/or position 25 as measured from the initial catalytic cysteine and wherein the substituted amino acid provides increased solubility in aqueous buffers compared to the native N-intein protein sequence or a consensus N-intein sequence. The invention also encompasses inteins which have a naturally occurring E in position 24, such as N-inteins from Limnorafis robusta.

Preferably the substituted amino acid(s) that provide increased solubility is a non-positive amino acid. In a preferred embodiment the substituted amino acid that provide increased solubility is K24E. In another embodiment the substituted amino acid that provide increased solubility is R25N. In a most preferred embodiment the N-inten comprises both these mutations.

The invention relates to an N-intein protein variant of the wildtype N-intein domain of Nostoc punctiforme (Npu) wherein the wildtype Npu N-intein domain comprises the following sequence: CLSYETEILTVEYGLLPIGKIVEKRIECTVYSVDNNGNIYTQPVAQWHDRGEQEVFEY CLEDGSLIRATKDHKFMTVDGQMLPIDEIFERELDLMRV (SEQ ID NO 1) (construct A52 in the Examples), wherein the protein variant comprises an amino acid substitution from K to E in position 24 and R to N in position 25 (construct B97 in the Examples) to increase solubility in aqueous buffers to minimize formation of inclusion bodies, wherein optionally one or more C (Cys) are mutated to non-Cystein residues, preferably S (Ser) or A (Ala). Further constructs encompassed by the invention are described in the example section below.

The N-intein protein variant as described above has solubility in aqueous buffer of at least 10-40% soluble N-intein with a single-point mutation of R at position 25, preferred N or non-positive amino acid; at least 46-52% soluble N-intein with a single-point mutation of K at position 24, preferred E or non-positive amino acid; and at least 76-88% soluble N-intein with mutations at positions 24 and 25, preferred K24E and R25N or non-positive amino acids.

The N-intein variant may be coupled to solid phase, such as a membrane, fiber, particle, bead or chip, such as chromatography resin of natural or synthetic origin.

The solid phase may optionally be provided with embedded magnetic particles. In an alternative embodiment the solid phase is a non-diffusion limited resin/fibrous material. According to the invention 0.2-2 μmole/ml N-intein is coupled per ml solid phase, preferably chromatography resin (ml swollen gel).

In a second aspect the invention relates to a split intein system comprising a N-intein as described above and a C-intein sequence which is co-expressed with a POI (protein of interest). The C-intein acts as a tag on the POI for binding to the N-intein attached to solid phase. After binding, the POI is cleaved of from the combined N-intein and C-intein and delivering a tagless POI. The C-intein variant is a split intein C-intein sequence or engineered variants thereof. A preferred C-intein sequence is mentioned in WO2021/099607 A1.

The POI's may be any recombinant proteins: proteins requiring native or near native N-terminal sequences, for example therapeutic protein candidates, biologics, antibody fragments, antibody mimetics, enzymes, recombinant proteins or peptides, such as growth factors, cytokines, chemokines, hormones, antigen (viral, bacterial, yeast, mammalian) production, vaccine production, cell surface receptors, fusion proteins.

### BRIEF DESCRIPTION OF THE DRAWINGS

FIG. 1 shows a SDS-PAGE analysis of supernatants from different constructs after extraction using different techniques.

FIG. 2 shows solubility of different constructs determined after densitometric evaluation of SDS-PAGE analysis. Extracts from three different cell-cultures for each construct were analysed. Bars show the average solubility compared with whole cell lysate, (SDS) and the error bars show the standard deviation.

FIG. 3 shows N-intein concentrations in supernatants from different extracts determined by Biacore CFCA analysis. Extracts from three different cell-cultures for each construct were analysed. Bars show the average concentration and the error bars show the standard deviation.

FIG. 4 shows the ratio of N-intein in supernatants from extracts of different constructs using different extraction methods, compared as % to total amount of N-intein solubilized by SDS and heating.

### DETAILED DESCRIPTION OF THE INVENTION

### Definitions

As used in the specification and the appended claims, the singular forms “a,” “an” and “the” include plural referents unless the context clearly dictates otherwise. Thus, for example, reference to “a functional group,” “an alkyl,” or “a residue” includes mixtures of two or more such functional groups, alkyls, or residues, and the like.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself.

A weight percent (wt. %) of a component, unless specifically stated to the contrary, is based on the total weight of the formulation or composition in which the component is included.

As used herein, the terms “optional” or “optionally” means that the subsequently described event or circumstance can or can not occur, and that the description includes instances where said event or circumstance occurs and instances where it does not.

The term “contacting” as used herein refers to bringing two biological entities together in such a manner that the compound can affect the activity of the target, either directly; i.e., by interacting with the target itself, or indirectly; i.e., by interacting with another molecule, co-factor, factor, or protein on which the activity of the target is dependent. “Contacting” can also mean facilitating the interaction of two biological entities, such as peptides, to bond covalently or otherwise.

The term “peptide”, “polypeptides” and “protein” are used interchangeably herein and include proteins and fragments thereof. Polypeptides are disclosed herein as amino acid residue sequences. Those sequences are written left to right in the direction from the amino to the carboxy terminus. In accordance with standard nomenclature, amino acid residue sequences are denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V). Peptides include any oligopeptide, polypeptide, gene product, expression product, or protein. A peptide is comprised of consecutive amino acids and encompasses naturally occurring or synthetic molecules.

In addition, as used herein, the term “peptide” refers to amino acids joined to each other by peptide bonds or modified peptide bonds, e.g., peptide isosteres, etc. and may contain modified amino acids other than the 20 gene-encoded amino acids. The peptides can be modified by either natural processes, such as post-translational processing, or by chemical modification techniques which are well known in the art. Modifications can occur anywhere in the peptide, including the peptide backbone, the amino acid side-chains and the amino or carboxyl termini. The same type of modification can be present in the same or varying degrees at several sites in a given polypeptide. Also, a given peptide can have many types of modifications. Modifications include, without limitation, linkage of distinct domains or motifs, acetylation, acylation, ADP-ribosylation, amidation, covalent cross-linking or cyclization, covalent attachment of flavin, covalent attachment of a heme moiety, covalent attachment of a nucleotide or nucleotide derivative, covalent attachment of a lipid or lipid derivative, covalent attachment of a phosphytidylinositol, disulfide bond formation, demethylation, formation of cysteine or pyroglutamate, formylation, gamma-carboxylation, glycosylation, GPI anchor formation, hydroxylation, iodination, methylation, myristolyation, oxidation, pergylation, proteolytic processing, phosphorylation, prenylation, racemization, selenoylation, sulfation, and transfer-RNA mediated addition of amino acids to protein such as arginylation. (See Proteins—Structure and Molecular Properties 2nd Ed., T. E. Creighton, W. H. Freeman and Company, New York (1993); Posttranslational Covalent Modification of Proteins, B. C. Johnson, Ed., Academic Press, New York, pp. 1-12 (1983)).

As used herein, “variant” refers to a molecule that retains a biological activity that is the same or substantially similar to that of the original sequence. The variant may be from the same or different species or be a synthetic sequence based on a natural or prior molecule. Moreover, as used herein, “variant” refers to a molecule having a structure attained from the structure of a parent molecule (e.g., a protein or peptide disclosed herein) and whose structure or sequence is sufficiently similar to those disclosed herein that based upon that similarity, would be expected by one skilled in the art to exhibit the same or similar activities and utilities compared to the parent molecule. For example, substituting specific amino acids in a given peptide can yield a variant peptide with similar activity to the parent.

In the context of the present invention, a substitution in a variant protein is indicated as: [original amino acid/position in sequence/substituted amino acid].

As used herein, the term “protein of interest (POI)” includes any synthetic or naturally occurring protein or peptide. The term therefore encompasses those compounds traditionally regarded as drugs, vaccines, and biopharmaceuticals including molecules such as proteins, peptides, and the like. Examples of therapeutic agents are described in well-known literature references such as the Merck Index (14th edition), the Physicians' Desk Reference (64th edition), and The Pharmacological Basis of Therapeutics (1st edition), and they include, without limitation, medicaments; substances used for the treatment, prevention, diagnosis, cure or mitigation of a disease or illness; substances that affect the structure or function of the body, or pro-drugs, which become biologically active or more active after they have been placed in a physiological environment.

As used herein, “isolated peptide” or “purified peptide” is meant to mean a peptide (or a fragment thereof) that is substantially free from the materials with which the peptide is normally associated in nature, or from the materials with which the peptide is associated in an artificial expression or production system, including but not limited to an expression host cell lysate, growth medium components, buffer components, cell culture supernatant, or components of a synthetic in vitro translation system. The peptides disclosed herein, or fragments thereof, can be obtained, for example, by extraction from a natural source (for example, a mammalian cell), by expression of a recombinant nucleic acid encoding the peptide (for example, in a cell or in a cell-free translation system), or by chemically synthesizing the peptide. In addition, peptide fragments may be obtained by any of these methods, or by cleaving full length proteins and/or peptides.

The word “or” as used herein means any one member of a particular list and also includes any combination of members of that list.

The phrase “nucleic acid” as used herein refers to a naturally occurring or synthetic oligonucleotide or polynucleotide, whether DNA or RNA or DNA-RNA hybrid, single-stranded or double-stranded, sense or antisense, which is capable of hybridization to a complementary nucleic acid by Watson-Crick base-pairing. Nucleic acids of the invention can also include nucleotide analogs (e.g., BrdU), and non-phosphodiester internucleoside linkages (e.g., peptide nucleic acid (PNA) or thiodiester linkages). In particular, nucleic acids can include, without limitation, DNA, RNA, cDNA, gDNA, ssDNA, dsDNA or any combination thereof.

As used herein, “extein” refers to the portion of an intein-modified protein that is not part of the intein and which can be spliced or cleaved upon excision of the intein.

“Intein” refers to an in-frame intervening sequence in a protein. An intein can catalyze its own excision from the protein through a post-translational protein splicing process to yield the free intein and a mature protein. An intein can also catalyze the cleavage of the intein-extein bond at either the intein N-terminus, or the intein C-terminus, or both of the intein-extein termini. As used herein, “intein” encompasses mini-inteins, modified or mutated inteins, and split inteins.

As used herein, the term “split intein” refers to any intein in which one or more peptide bond breaks exists between the N-terminal intein segment and the C-terminal intein segment such that the N-terminal and C-terminal intein segments become separate molecules that can non-covalently reassociate, or reconstitute, into an intein that is functional for splicing or cleaving reactions. Any catalytically active intein, or fragment thereof, may be used to derive a split intein for use in the systems and methods disclosed herein. For example, in one aspect the split intein may be derived from a eukaryotic intein. In another aspect, the split intein may be derived from a bacterial intein. In another aspect, the split intein may be derived from an archaeal intein. Preferably, the split intein so-derived will possess only the amino acid sequences essential for catalyzing splicing reactions.

As used herein, the “N-terminal intein segment” or “N-intein” refers to any intein sequence that comprises an N-terminal amino acid sequence that is functional for splicing and/or cleaving reactions when combined with a corresponding C-terminal intein segment. An N-terminal intein segment thus also comprises a sequence that is spliced out when splicing occurs. An N-terminal intein segment can comprise a sequence that is a modification of the N-terminal portion of a naturally occurring (native) intein sequence. Non-intein residues can also be genetically fused to intein segments to provide additional functionality, such as the ability to be affinity purified or to be covalently immobilized.

As used herein, the “C-terminal intein segment” or “C-intein” refers to any intein sequence that comprises a C-terminal amino acid sequence that is functional for splicing or cleaving reactions when combined with a corresponding N-terminal intein segment. In one aspect, the C-terminal intein segment comprises a sequence that is spliced out when splicing occurs. In another aspect, the C-terminal intein segment is cleaved from a peptide sequence fused to its C-terminus. The sequence which is cleaved from the C-terminal intein's C-terminus is referred to herein as a “protein of interest POI” is discussed in more detail below. A C-terminal intein segment can comprise a sequence that is a modification of the C-terminal portion of a naturally occurring (native) intein sequence. For example, a C terminal intein segment can comprise additional amino acid residues and/or mutated residues so long as the inclusion of such additional and/or mutated residues does not render the C-terminal intein segment non-functional for splicing or cleaving.

A consensus sequence is a sequence of DNA, RNA, or protein that represents aligned, related sequences. The consensus sequence of the related sequences can be defined in different ways, but is normally defined by the most common nucleotide(s) or amino acid residue(s) at each position.

As used herein, the term “splice” or “splices” means to excise a central portion of a polypeptide to form two or more smaller polypeptide molecules. In some cases, splicing also includes the step of fusing together two or more of the smaller polypeptides to form a new polypeptide. Splicing can also refer to the joining of two polypeptides encoded on two separate gene products through the action of a split intein.

As used herein, the term “cleave” or “cleaves” means to divide a single polypeptide to form two or more smaller polypeptide molecules. In some cases, cleavage is mediated by the addition of an extrinsic endopeptidase, which is often referred to as “proteolytic cleavage”. In other cases, cleaving can be mediated by the intrinsic activity of one or both of the cleaved peptide sequences, which is often referred to as “self-cleavage”. Cleavage can also refer to the self-cleavage of two polypeptides that is induced by the addition of a non-proteolytic third peptide, as in the action of split intein system described herein.

By the term “fused” is meant covalently bonded to. For example, a first peptide is fused to a second peptide when the two peptides are covalently bonded to each other (e.g., via a peptide bond).

As used herein an “isolated” or “substantially pure” substance is one that has been separated from components which naturally accompany it. Typically, a polypeptide is substantially pure when it is at least 50% (e.g., 60%, 70%, 80%, 90%, 95%, and 99%) by weight free from the other proteins and naturally-occurring organic molecules with which it is naturally associated.

Herein, “bind” or “binds” means that one molecule recognizes and adheres to another molecule in a sample, but does not substantially recognize or adhere to other molecules in the sample. One molecule “specifically binds” another molecule if it has a binding affinity greater than about 10⁵ to 10⁶ liters/mole for the other molecule.

Nucleic acids, nucleotide sequences, proteins or amino acid sequences referred to herein can be isolated, purified, synthesized chemically, or produced through recombinant DNA technology. All of these methods are well known in the art.

As used herein, the terms “modified” or “mutated,” as in “modified intein” or “mutated intein,” refer to one or more modifications in either the nucleic acid or amino acid sequence being referred to, such as an intein, when compared to the native, or naturally occurring structure. Such modification can be a substitution, addition, or deletion. The modification can occur in one or more amino acid residues or one or more nucleotides of the structure being referred to, such as an intein.

As used herein, the term “modified peptide”, “modified protein” or “modified protein of interest” or “modified target protein” refers to a protein which has been modified.

As used herein, “operably linked” refers to the association of two or more biomolecules in a configuration relative to one another such that the normal function of the biomolecules can be performed. In relation to nucleotide sequences, “operably linked” refers to the association of two or more nucleic acid sequences, by means of enzymatic ligation or otherwise, in a configuration relative to one another such that the normal function of the sequences can be performed. For example, the nucleotide sequence encoding a pre-sequence or secretory leader is operably linked to a nucleotide sequence for a polypeptide if it is expressed as a pre-protein that participates in the secretion of the polypeptide; a promoter or enhancer is operably linked to a coding sequence if it affects the transcription of the coding sequence; and a ribosome binding site is operably linked to a coding sequence if it is positioned so as to facilitate translation of the sequence.

“Sequence homology” can refer to the situation where nucleic acid or protein sequences are similar because they have a common evolutionary origin. “Sequence homology” can indicate that sequences are very similar. Sequence similarity is observable; homology can be based on the observation. “Very similar” can mean at least 70% identity, homology or similarity; at least 75% identity, homology or similarity; at least 80% identity, homology or similarity; at least 85% identity, homology or similarity; at least 90% identity, homology or similarity; such as at least 93% or at least 95% or even at least 97% identity, homology or similarity. The nucleotide sequence similarity or homology or identity can be determined using the “Align” program of Myers et al. (1988) CABIOS 4:11-17 and available at NCBI. Additionally or alternatively, amino acid sequence similarity or identity or homology can be determined using the BlastP program (Altschul et al. Nucl. Acids Res. 25:3389-3402), and available at NCBI. Alternatively or additionally, the terms “similarity” or “identity” or “homology,” for instance, with respect to a nucleotide sequence, are intended to indicate a quantitative measure of homology between two sequences.

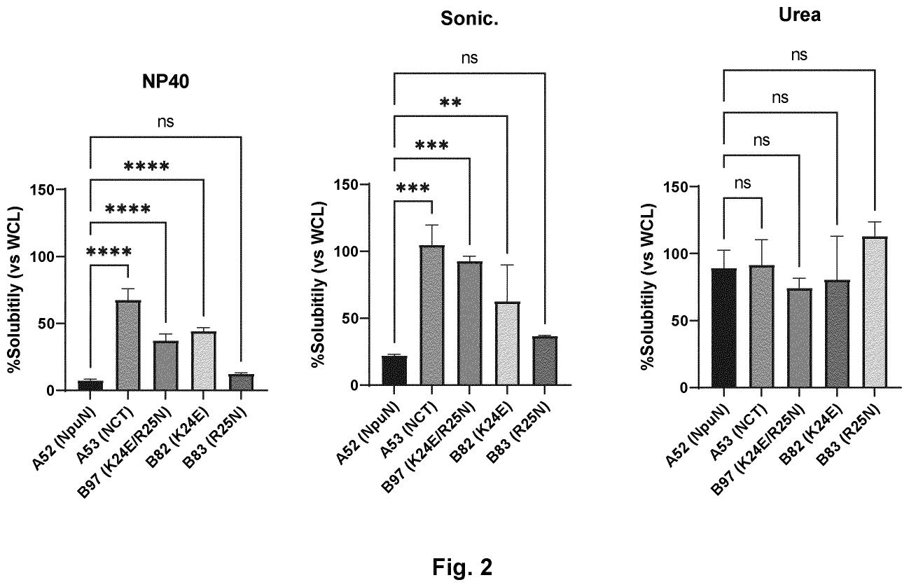

Alternatively or additionally, “similarity” with respect to sequences refers to the number of positions with identical nucleotides divided by the number of nucleotides in the shorter of the two sequences wherein alignment of the two sequences can be determined in accordance with the Wilbur and Lipman algorithm. (1983) Proc. Natl. Acad. Sci. USA 80:726. For example, using a window size of 20 nucleotides, a word length of 4 nucleotides, and a gap penalty of 4, and computer-assisted analysis and interpretation of the sequence data including alignment can be conveniently performed using commercially available programs (e.g., Intelligenetics™ Suite, Intelligenetics™ Inc. CA). When RNA sequences are said to be similar, or have a degree of sequence identity with DNA sequences, thymidine (T) in the DNA sequence is considered equal to uracil (U) in the RNA sequence. The following references also provide algorithms for comparing the relative identity or homology or similarity of amino acid residues of two proteins, and additionally or alternatively with respect to the foregoing, the references can be used for determining percent homology or identity or similarity. Needleman et al. (1970) J. Mol. Biol. 48:444-453; Smith et al. (1983) Advances App. Math. 2:482-489; Smith et al. (1981) Nuc. Acids Res. 11:2205-2220; Feng et al. (1987) J. Molec. Evol. 25:351-360; Higgins et al. (1989) CABIOS 5:151-153; Thompson et al. (1994) Nuc. Acids Res. 22:4673-480; and Devereux et al. (1984) 12:387-395. “Stringent hybridization conditions” is a term which is well known in the art; see, for example, Sambrook, “Molecular Cloning, A Laboratory Manual” second ed., CSH Press, Cold Spring Harbor, 1989; “Nucleic Acid Hybridization, A Practical Approach”, Hames and Higgins eds., IRL Press, Oxford, 1985; see also FIG. 2 and description thereof herein wherein there is a sequence comparison.

The term “buffer” or “buffered solution” refers to solutions which resist changes in pH by the action of its conjugate acid-base range.

The term “loading buffer” or “equilibrium buffer” refers to the buffer containing the salt or salts which is mixed with the protein preparation for loading the protein preparation onto a column. This buffer is also used to equilibrate the column before loading, and to wash to column after loading the protein.

The term “wash buffer” is used herein to refer to the buffer that is passed over a column (for example) following loading of a protein of interest (such as one coupled to a C-terminal intein fragment, for example) and prior to elution of the protein of interest. The wash buffer may serve to remove one or more contaminants without substantial elution of the desired protein.

The term “elution buffer” refers to the buffer used to elute the desired protein from the column. As used herein, the term “solution” refers to either a buffered or a non-buffered solution, including water.

The term “washing” means passing an appropriate buffer through or over a solid support, such as a chromatographic resin.

The term “eluting” a molecule (e.g. a desired protein or contaminant) from a solid support means removing the molecule from such material.

The term “contaminant” or “impurity” refers to any foreign or objectionable molecule, particularly a biological macromolecule such as a DNA, an RNA, or a protein, other than the protein being purified, that is present in a sample of a protein being purified. Contaminants include, for example, other proteins from cells that express and/or secrete the protein being purified.

The term “separate” or “isolate” as used in connection with protein purification refers to the separation of a desired protein from a second protein or other contaminant or mixture of impurities in a mixture comprising both the desired protein and a second protein or other contaminant or impurity mixture, such that at least the majority of the molecules of the desired protein are removed from that portion of the mixture that comprises at least the majority of the molecules of the second protein or other contaminant or mixture of impurities.

The term “purify” or “purifying” a desired protein from a composition or solution comprising the desired protein and one or more contaminants means increasing the degree of purity of the desired protein in the composition or solution by removing (completely or partially) at least one contaminant from the composition or solution.

### N-Intein Protein Variants

The invention relates to affinity chromatography and affinity tag cleavage mechanisms in a single step using a split intein system according to the invention which cleaves with broad amino acid tolerance to generate a tag less protein of interest (POI) as end product. The two halves of the intein are the affinity ligand (N-intein) and the affinity tag (C-intein) and they associate rapidly. Immobilizing one half (N-intein) on a chromatography resin enables the capture of the other half (C-intein) coupled to the POI from solution. In the presence of Zn²⁺ ions, the cleavage reaction is inhibited, enabling a stable complex to form while impurities are washed away. After impurities are eliminated, a chelator or reducing agent is added, and the cleavage reaction proceeds, enabling collection of the POI, while the intein tag remains bound non-covalently to the cognate intein linked to the chromatography resin.

Preferably the invention provides N-intein protein variant sequences of native split inteins or consensus sequences derived from native inteins and split inteins wherein, the N-intein variant is modified as compared to the native sequence or consensus sequence to provide increased solubility by having mutations in position 24 and/or 25. These positions are calculated according to conventional clustal alignment with native split inteins starting from the initial catalytical cysteine which is number 1.

Native intein are known in the art. A list of inteins is found in Table 1 below. All inteins have the potential to be made into split inteins while some inteins naturally exist in split form. All of the inteins found in the table either exist as split inteins or have the potential to be made into split inteins modified in accordance with the invention at position 24 and/or 25 such that the increased solubility is achieved compared to the native sequences.

TABLE 1
Naturally Occurring Inteins

| Intein Name | Organism Name | Organism Description |
|---|---|---|
| Eucarya | | |
| APMV Pol | Acanthomoeba polyphaga Mimivirus | isolate = “Rowbotham- |
| | | Bradford”, Virus, infects |
| | | Amoebae, taxon: 212035 |
| Abr PRP8 | Aspergillus brevipes FRR2439 | Fungi, ATCC 16899, |
| | | taxon: 75551 |
| Aca-G186AR PRP8 | Ajellomyces capsulatus G186AR | Taxon: 447093, strain |
| | | G186AR |
| Aca-H143 PRP8 | Ajellomyces capsulatus H143 | Taxon: 544712 |
| Aca-JER2004 PRP8 | Ajellomyces capsulatus (anamorph: | strain = JER2004, taxon: 5037, |
| | Histoplasma capsulatum) | Fungi |
| Aca-NAm1 PRP8 | Ajellomyces capsulatus NAm1 | strain = “NAm1”, taxon: 339724 |
| Ade-ER3 PRP8 | Ajellomyces dermatitidis ER-3 | Human fungal |
| | | pathogen. taxon: 559297 |
| Ade-SLH14081 PRP8 | Ajellomyces dermatitidis SLH14081, | Human fungal pathogen |
| Afu-Af293 PRP8 | Aspergillus fumigatus var. ellipticus, | Human pathogenic fungus, |
| | strain Af293 | taxon: 330879 |
| Afu-FRR0163 PRP8 | Aspergillus fumigatus strain | Human pathogenic fungus, |
| | FRR0163 | taxon: 5085 |
| Afu-NRRL5109 PRP8 | Aspergillus fumigatus var. ellipticus, | Human pathogenic fungus, |
| | strain NRRL 5109 | taxon: 41121 |
| Agi-NRRL6136 PRP8 | Aspergillus giganteus Strain NRRL | Fungus, taxon: 5060 |
| | 6136 | |
| Ani-FGSCA4 PRP8 | Aspergillus nidulans FGSC A | Filamentous fungus, |
| | | taxon: 227321 |
| Avi PRP8 | Aspergillus viridinutans strain | Fungi, ATCC 16902, |
| | FRR0577 | taxon: 75553 |
| Bci PRP8 | Botrytis cinerea (teleomorph of | Plant fungal pathogen |
| | Botryotinia fuckeliana B05.10) | |
| Bde-JEL197 RPB2 | Batrachochytrium dendrobatidis | Chytrid fungus, |
| | JEL197 | isolate = “AFTOL-ID 21”, |
| | | taxon: 109871 |
| Bde-JEL423 PRP8-1 | Batrachochytrium dendrobatidis | Chytrid fungus, isolate |
| | JEL423 | JEL423, taxon 403673 |
| Bde-JEL423 PRP8-2 | Batrachochytrium dendrobatidis | Chytrid fungus, isolate |
| | JEL423 | JEL423, taxon 403673 |
| Bde-JEL423 RPC2 | Batrachochytrium dendrobatidis | Chytrid fungus, isolate |
| | JEL423 | JEL423, taxon 403673 |
| Bde-JEL423 eIF-5B | Batrachochytrium dendrobatidis | Chytrid fungus, isolate |
| | JEL423 | JEL423, taxon 403673 |
| Bfu-B05 PRP8 | Botryotinia fuckeliana B05.10 | Taxon: 332648 |
| CIV RIR1 | Chilo iridescent virus | dsDNA eucaryotic virus, |
| | | taxon: 10488 |
| CV-NY2A ORF212392 | Chlorella virus NY2A infects | dsDNA eucaryotic |
| | Chlorella NC64A, which infects | virus, taxon: 46021, Family |
| | Paramecium bursaria | Phycodnaviridae |
| CV-NY2A RIR1 | Chlorella virus NY2A infects | dsDNA eucaryotic |
| | Chlorella NC64A, which infects | virus, taxon: 46021, Family |
| | Paramecium bursaria | Phycodnaviridae |
| CZIV RIR1 | Costelytra zealandica iridescent virus | dsDNA eucaryotic virus, |
| | | Taxon: 68348 |
| Cba-WM02.98 PRP8 | Cryptococcus bacillisporus strain | Yeast, human pathogen, |
| | WM02.98 (aka Cryptococcus | taxon: 37769 |
| | neoformans gattii) | |
| Cba-WM728 PRP8 | Cryptococcus bacillisporus strain | Yeast, human pathogen, |
| | WM728 | taxon: 37769 |
| Ceu ClpP | Chlamydomonas eugametos | Green alga, taxon: 3053 |
| | (chloroplast) | |
| Cga PRP8 | Cryptococcus gattii (aka | Yeast, human pathogen |
| | Cryptococcus bacillisporus) | |
| Cgl VMA | Candida glabrata | Yeast, taxon: 5478 |
| Cla PRP8 | Cryptococcus laurentii strain | Fungi, Basidiomycete yeast, |
| | CBS139 | taxon: 5418 |
| Cmo ClpP | Chlamydomonas moewusii, strain | Green alga, chloroplast gene, |
| | UTEX 97 | taxon: 3054 |
| Cmo RPB2 (RpoBb) | Chlamydomonas moewusii, strain | Green alga, chloroplast gene, |
| | UTEX 97 | taxon: 3054 |
| Cne-A PRP8 (Fne-A | Filobasidiella neoformans | Yeast, human pathogen |
| PRP8) | (Cryptococcus neoformans) Serotype | |
| | A, PHLS_8104 | |
| Cne-AD PRP8 (Fne- | Cryptococcus neoformans | Yeast, human pathogen, |
| AD PRP8) | (Filobasidiella neoformans), | ATCC32045, taxon: 5207 |
| | Serotype AD, CBS132). | |
| Cne-JEC21 PRP8 | Cryptococcus neoformans var. | Yeast, human pathogen, |
| | neoformans JEC21 | serotype = “D” taxon: 214684 |
| Cpa ThrRS | Candida parapsilosis, strain CLIB214 | Yeast, Fungus, taxon: 5480 |
| Cre RPB2 | Chlamydomonas reinhardtii | Green algae, taxon: 3055 |
| | (nucleus) | |
| CroV Pol | Cafeteria roenbergensis virus BV- | taxon: 693272, Giant virus |
| | PW1 | infecting marine heterotrophic |
| | | nanoflagellate |
| CroV RIR1 | Cafeteria roenbergensis virus BV- | taxon: 693272, Giant virus |
| | PW1 | infecting marine heterotrophic |
| | | nanoflagellate |
| CroV RPB2 | Cafeteria roenbergensis virus BV- | taxon: 693272, Giant virus |
| | PW1 | infecting marine heterotrophic |
| | | nanoflagellate |
| CroV Top2 | Cafeteria roenbergensis virus BV- | taxon: 693272, Giant virus |
| | PW1 | infecting marine heterotrophic |
| | | nanoflagellate |
| Cst RPB2 | Coelomomyces stegomyiae | Chytrid fungus, |
| | | isolate = “AFTOL-ID 18”, |
| | | taxon: 143960 |
| Ctr ThrRS | Candida tropicalis ATCC750 | Yeast |
| Ctr VMA | Candida tropicalis (nucleus) | Yeast |
| Ctr-MYA3404 VMA | Candida tropicalis MYA-3404 | Taxon: 294747 |
| Ddi RPC2 | Dictyostelium discoideum strain | Mycetozoa (a social amoeba) |
| | AX4 (nucleus) | |
| Dhan GLT1 | Debaryomyces hansenii CBS767 | Fungi, Anamorph: Candida |
| | | famata, taxon: 4959 |
| Dhan VMA | Debaryomyces hansenii CBS767 | Fungi, taxon: 284592 |
| Eni PRP8 | Emericella nidulans R20 (anamorph: | taxon: 162425 |
| | Aspergillus nidulans) | |
| Eni-FGSCA4 PRP8 | Emericella nidulans (anamorph: | Filamentous fungus, |
| | Aspergillus nidulans) FGSC A4 | taxon: 162425 |
| Fte RPB2 (RpoB) | Floydiella terrestris, strain UTEX | Green alga, chloroplast gene, |
| | 1709 | taxon: 51328 |
| Gth DnaB | Guillardia theta (plastid) | Cryptophyte Algae |
| HaV01 Pol | Heterosigma akashiwo virus 01 | Algal virus, taxon: 97195, |
| | | strain HaV01 |
| Hca PRP8 | Histoplasma capsulatum (anamorph: | Fungi, human pathogen |
| | Ajellomyces capsulatus) | |
| IIV6 RIR1 | Invertebrate iridescent virus 6 | dsDNA eucaryotic |
| | | virus, taxon: 176652 |
| Kex-CBS379 VMA | Kazachstania exigua, formerly | Yeast, taxon: 34358 |
| | Saccharomyces exiguus, strain | |
| | CBS379 | |
| Kla-CBS683 VMA | Kluyveromyces lactis, strain CBS683 | Yeast, taxon: 28985 |
| Kla-IFO1267 VMA | Kluyveromyces lactis IFO1267 | Fungi, taxon: 28985 |
| Kla-NRRLY1140 VMA | Kluyveromyces lactis NRRL Y-1140 | Fungi, taxon: 284590 |
| Lel VMA | Lodderomyces elongisporus | Yeast |
| Mca-CBS113480 PRP8 | Microsporum canis CBS 113480 | Taxon: 554155 |
| Nau PRP8 | Neosartorya aurata NRRL 4378 | Fungus, taxon: 41051 |
| Nfe-NRRL5534 PRP8 | Neosartorya fennelliae NRRL 5534 | Fungus, taxon: 41048 |
| Nfi PRP8 | Neosartorya fischeri | Fungi |
| Ngl-FR2163 PRP8 | Neosartorya glabra FRR2163 | Fungi, ATCC 16909, |
| | | taxon: 41049 |
| Ngl-FRR1833 PRP8 | Neosartorya glabra FRR1833 | Fungi, taxon: 41049, |
| | | (preliminary identification) |
| Nqu PRP8 | Neosartorya quadricincta, strain | taxon: 41053 |
| | NRRL 4175 | |
| Nspi PRP8 | Neosartorya spinosa FRR4595 | Fungi, taxon: 36631 |
| Pabr-Pb01 PRP8 | Paracoccidioides brasiliensis Pb01 | Taxon: 502779 |
| Pabr-Pb03 PRP8 | Paracoccidioides brasiliensis Pb03 | Taxon: 482561 |
| Pan CHS2 | Podospora anserina | Fungi, Taxon 5145 |
| Pan GLT1 | Podospora anserina | Fungi, Taxon 5145 |
| Pbl PRP8-a | Phycomyces blakesleeanus | Zygomycete fungus, strain |
| | | NRRL155 |
| Pbl PRP8-b | Phycomyces blakesleeanus | Zygomycete fungus, strain |
| | | NRRL155 |
| Pbr-Pb18 PRP8 | Paracoccidioides brasiliensis Pb18 | Fungi, taxon: 121759 |
| Pch PRP8 | Penicillium chrysogenum | Fungus, taxon: 5076 |
| Pex PRP8 | Penicillium expansum | Fungus, taxon27334 |
| Pgu GLT1 | Pichia (Candida) guilliermondii | Fungi, Taxon 294746 |
| Pgu-alt GLT1 | Pichia (Candida) guilliermondii | Fungi |
| Pno GLT1 | Phaeosphaeria nodorum SN15 | Fungi, taxon: 321614 |
| Pno RPA2 | Phaeosphaeria nodorum SN15 | Fungi, taxon: 321614 |
| Ppu DnaB | Porphyra purpurea (chloroplast) | Red Alga |
| Pst VMA | Pichia stipitis CBS 6054, | Yeast |
| | taxon: 322104 | |
| Ptr PRP8 | Pyrenophora tritici-repentis Pt-1C- | Ascomycete |
| | BF | fungus, taxon: 426418 |
| Pvu PRP8 | Penicillium vulpinum (formerly | Fungus |
| | P. claviforme) | |
| Pye DnaB | Porphyra yezoensis chloroplast, | Red alga, |
| | cultivar U-51 | organelle = “plastid: chloroplast”, |
| | | “taxon: 2788 |
| Sas RPB2 | Spiromyces aspiralis NRRL 22631 | Zygomycete fungus, |
| | | isolate = “AFTOL-ID |
| | | 185”, taxon: 68401 |
| Sca-CBS4309 VMA | Saccharomyces castellii, strain | Yeast, taxon: 27288 |
| | CBS4309 | |
| Sca-IFO1992 VMA | Saccharomyces castellii, strain | Yeast, taxon: 27288 |
| | IFO1992 | |
| Scar VMA | Saccharomyces cariocanus, | Yeast, taxon: 114526 |
| | strain = “UFRJ 50791 | |
| Sce VMA | Saccharomyces cerevisiae (nucleus) | Yeast, also in Sce strains |
| | | OUT7163, OUT7045, |
| | | OUT7163, IFO1992 |
| Sce-DH1-1A VMA | Saccharomyces cerevisiae strain | Yeast, taxon: 173900, also in |
| | DH1-1A | Sce strains |
| | | OUT7900, OUT7903, OUT7112 |
| Sce-JAY291 VMA | Saccharomyces cerevisiae JAY291 | Taxon: 574961 |
| Sce-OUT7091 VMA | Saccharomyces cerevisiae OUT7091 | Yeast, taxon: 4932, also in Sce |
| | | strains OUT7043, OUT7064 |
| Sce-OUT7112 VMA | Saccharomyces cerevisiae OUT7112 | Yeast, taxon: 4932, also in Sce |
| | | strains OUT7900, OUT7903 |
| Sce-YJM789 VMA | Saccharomyces cerevisiae strain | Yeast, taxon: 307796 |
| | YJM789 | |
| Sda VMA | Saccharomyces dairenensis, strain | Yeast, taxon: 27289, Also in |
| | CBS 421 | Sda strain IFO0211 |
| Sex-IFO1128 VMA | Saccharomyces exiguus, | Yeast, taxon: 34358 |
| | strain = “IFO1128” | |
| She RPB2 (RpoB) | Stigeoclonium helveticum, strain | Green alga, chloroplast gene, |
| | UTEX 441 | taxon: 55999 |
| Sja VMA | Schizosaccharomyces japonicus | Ascomycete fungus, |
| | yFS275 | taxon: 402676 |
| Spa VMA | Saccharomyces pastorianus | Yeast, taxon: 27292 |
| | IFO11023 | |
| Spu PRP8 | Spizellomyces punctatus | Chytrid fungus, |
| Sun VMA | Saccharomyces unisporus, strain | Yeast, taxon: 27294 |
| | CBS 398 | |
| Tgl VMA | Torulaspora globosa, strain CBS 764 | Yeast, taxon: 48254 |
| Tpr VMA | Torulaspora pretoriensis, strain CBS | Yeast, taxon: 35629 |
| | 5080 | |
| Ure-1704 PRP8 | Uncinocarpus reesii | Filamentous fungus |
| Vpo VMA | Vanderwaltozyma polyspora, | Yeast, taxon: 36033 |
| | formerly Kluyveromyces polysporus, | |
| | strain CBS 2163 | |
| WIV RIR1 | Wiseana iridescent virus | dsDNA eucaryotic |
| | | virus, taxon: 68347 |
| Zba VMA | Zygosaccharomyces bailii, strain | Yeast, taxon: 4954 |
| | CBS 685 | |
| Zbi VMA | Zygosaccharomyces bisporus, strain | Yeast, taxon: 4957 |
| | CBS 702 | |
| Zro VMA | Zygosaccharomyces rouxii, strain | Yeast, taxon: 4956 |
| | CBS 688 | |
| Eubacteria | | |
| AP-APSE1 dpol | Acyrthosiphon pisum secondary | Bacteriophage, taxon: 67571 |
| | endosymbiot phage 1 | |
| AP-APSE2 dpol | Bacteriophage APSE-2, isolate = T5A | Bacteriophage of Candidatus |
| | | Hamiltonella defensa, |
| | | endosymbiot of |
| | | Acyrthosiphon pisum, |
| | | taxon: 340054 |
| AP-APSE4 dpol | Bacteriophage of Candidatus | Bacteriophage, taxon: 568990 |
| | Hamiltonella defensa strain 5ATac, | |
| | endosymbiot of Acyrthosiphon | |
| | pisum | |
| AP-APSE5 dpol | Bacteriophage APSE-5 | Bacteriophage of Candidatus |
| | | Hamiltonella defensa, |
| | | endosymbiot of Uroleucon |
| | | rudbeckiae, taxon: 568991 |
| AP-Aaphi23 MupF | Bacteriophage Aaphi23, | Actinobacillus |
| | Haemophilus phage Aaphi23 | actinomycetemcomitans |
| | | Bacteriophage, taxon: 230158 |
| Aae RIR2 | Aquifex aeolicus strain VF5 | Thermophilic |
| | | chemolithoautotroph, |
| | | taxon: 63363 |
| Aave-AAC001 | Acidovorax avenae subsp. citrulli | taxon: 397945 |
| Aave1721 | AAC00-1 | |
| Aave-AAC001 RIR1 | Acidovorax avenae subsp. citrulli | taxon: 397945 |
| | AAC00-1 | |
| Aave-ATCC19860 | Acidovorax avenae subsp. avenae | Taxon: 643561 |
| RIR1 | ATCC 19860 | |
| Aba Hyp-02185 | Acinetobacter baumannii ACICU | taxon: 405416 |
| Ace RIR1 | Acidothermus cellulolyticus 11B | taxon: 351607 |
| Aeh DnaB-1 | Alkalilimnicola ehrlichei MLHE-1 | taxon: 187272 |
| Aeh DnaB-2 | Alkalilimnicola ehrlichei MLHE-1 | taxon: 187272 |
| Aeh RIR1 | Alkalilimnicola ehrlichei MLHE-1 | taxon: 187272 |
| AgP-S1249 MupF | Aggregatibacter phage S1249 | Taxon: 683735 |
| Aha DnaE-c | Aphanothece halophytica | Cyanobacterium, taxon: 72020 |
| Aha DnaE-n | Aphanothece halophytica | Cyanobacterium, taxon: 72020 |
| Alvi-DSM180 GyrA | Allochromatium vinosum DSM 180 | Taxon: 572477 |
| Ama MADE823 | phage uncharacterized protein | Probably prophage gene, |
| | [Alteromonas macleodii ‘Deep | taxon: 314275 |
| | ecotype’] | |
| Amax-CS328 DnaX | Arthrospira maxima CS-328 | Taxon: 513049 |
| Aov DnaE-c | Aphanizomenon ovalisporum | Cyanobacterium, taxon: 75695 |
| Aov DnaE-n | Aphanizomenon ovalisporum | Cyanobacterium, taxon: 75695 |
| Apl-C1 DnaX | Arthrospira platensis | Taxon: 118562, strain C1 |
| Arsp-FB24 DnaB | Arthrobacter species FB24 | taxon: 290399 |
| Asp DnaE-c | Anabaena species PCC7120, (Nostoc | Cyanobacterium, Nitrogen- |
| | sp. PCC7120) | fixing, taxon: 103690 |
| Asp DnaE-n | Anabaena species PCC7120, (Nostoc | Cyanobacterium, Nitrogen- |
| | sp. PCC7120) | fixing, taxon: 103690 |
| Ava DnaE-c | Anabaena variabilis ATCC29413 | Cyanobacterium, taxon: 240292 |
| Ava DnaE-n | Anabaena variabilis ATCC29413 | Cyanobacterium, taxon: 240292 |
| Avin RIR1 BIL | Azotobacter vinelandii | taxon: 354 |
| Bce-MCO3 DnaB | Burkholderia cenocepacia MC0-3 | taxon: 406425 |
| Bce-PC184 DnaB | Burkholderia cenocepacia PC184 | taxon: 350702 |
| Bse-MLS10 TerA | Bacillus selenitireducens MLS10 | Probably prophage gene, |
| | | Taxon: 439292 |
| BsuP-M1918 RIR1 | B. subtilis M1918 (prophage) | Prophage in B. subtilis M1918. |
| | | taxon: 157928 |
| BsuP-SPBc2 RIR1 | B. subtilis strain 168 Sp beta c2 | B. subtilis taxon 1423. SPbeta |
| | prophage | c2 phage, taxon: 66797 |
| Bvi IcmO | Burkholderia vietnamiensis G4 | plasmid = “pBVIE03”. |
| | | taxon: 269482 |
| CP-P1201 Thy1 | Corynebacterium phage P1201 | lytic bacteriophage P1201 |
| | | from Corynebacterium |
| | | glutamicum NCHU |
| | | 87078. Viruses; dsDNA |
| | | viruses, taxon: 384848 |
| Cag RIR1 | Chlorochromatium aggregatum | Motile, phototrophic consortia |
| Cau SpoVR | Chloroflexus aurantiacus J-10-fl | Anoxygenic |
| | | phototroph, taxon: 324602 |
| CbP-C-St RNR | Clostridium botulinum phage C-St | Phage, specific_host = “Clostridium |
| | | botulinum type C strain |
| | | C-Stockholm, taxon: 12336 |
| CbP-D1873 RNR | Clostridium botulinum phage D | Ssp. phage from Clostridium |
| | | botulinum type D strain, 1873, |
| | | taxon: 29342 |
| Cbu-Dugway DnaB | Coxiella burnetii Dugway 5J108-111 | Proteobacteria; Legionellales; |
| | | taxon: 434922 |
| Cbu-Goat DnaB | Coxiella burnetii ‘MSU Goat Q177’ | Proteobacteria; Legionellales; |
| | | taxon: 360116 |
| Cbu-RSA334 DnaB | Coxiella burnetii RSA 334 | Proteobacteria; Legionellales; |
| | | taxon: 360117 |
| Cbu-RSA493 DnaB | Coxiella burnetii RSA 493 | Proteobacteria; Legionellales; |
| | | taxon: 227377 |
| Cce Hyp1-Csp-2 | Cyanothece sp. ATCC 51142 | Marine unicellular |
| | | diazotrophic cyanobacterium, |
| | | taxon: 43989 |
| Cch RIR1 | Chlorobium chlorochromatii CaD3 | taxon: 340177 |
| Ccy Hyp1-Csp-1 | Cyanothece sp. CCY0110 | Cyanobacterium, |
| | | taxon: 391612 |
| Ccy Hyp1-Csp-2 | Cyanothece sp. CCY0110 | Cyanobacterium, |
| | | taxon: 391612 |
| Cfl-DSM20109 DnaB | Cellulomonas flavigena DSM 20109 | Taxon: 446466 |
| Chy RIR1 | Carboxydothermus | Thermophile, taxon = 246194 |
| | hydrogenoformans Z-2901 | |
| Ckl PTerm | Clostridium kluyveri DSM 555 | plasmid = “pCKL555A”, |
| | | taxon: 431943 |
| Cra-CS505 DnaE-c | Cylindrospermopsis raciborskii CS-505 | Taxon: 533240 |
| Cra-CS505 DnaE-n | Cylindrospermopsis raciborskii CS-505 | Taxon: 533240 |
| Cra-CS505 GyrB | Cylindrospermopsis raciborskii CS-505 | Taxon: 533240 |
| Csp-CCY0110 DnaE-c | Cyanothece sp. CCY0110 | Taxon: 391612 |
| Csp-CCY0110 DnaE-n | Cyanothece sp. CCY0110 | Taxon: 391612 |
| Csp-PCC7424 DnaE-c | Cyanothece sp. PCC 7424 | Cyanobacterium, taxon: 65393 |
| Csp-PCC7424 DnaE-n | Cyanothece sp. PCC7424 | Cyanobacterium, taxon: 65393 |
| Csp-PCC7425 DnaB | Cyanothece sp. PCC 7425 | Taxon: 395961 |
| Csp-PCC7822 DnaE-n | Cyanothece sp. PCC 7822 | Taxon: 497965 |
| Csp-PCC8801 DnaE-c | Cyanothece sp. PCC 8801 | Taxon: 41431 |
| Csp-PCC8801 DnaE-n | Cyanothece sp. PCC 8801 | Taxon: 41431 |
| Cth ATPase BIL | Clostridium thermocellum | ATCC27405, taxon: 203119 |
| Cth-ATCC27405 TerA | Clostridium thermocellum | Probable prophage, |
| | ATCC27405 | ATCC27405, taxon: 203119 |
| Cth-DSM2360 TerA | Clostridium thermocellum DSM | Probably prophage |
| | 2360 | gene, Taxon: 572545 |
| Cwa DnaB | Crocosphaera watsonii WH 8501 | taxon: 165597 |
| | (Synechocystis sp. WH 8501) | |
| Cwa DnaE-c | Crocosphaera watsonii WH 8501 | Cyanobacterium, |
| | (Synechocystis sp. WH 8501) | taxon: 165597 |
| Cwa DnaE-n | Crocosphaera watsonii WH 8501 | Cyanobacterium, |
| | (Synechocystis sp. WH 8501) | taxon: 165597 |
| Cwa PEP | Crocosphaera watsonii WH 8501 | taxon: 165597 |
| | (Synechocystis sp. WH 8501) | |
| Cwa RIR1 | Crocosphaera watsonii WH 8501 | taxon: 165597 |
| | (Synechocystis sp. WH 8501) | |
| Daud RIR1 | Candidatus Desulforudis audaxviator | taxon: 477974 |
| | MP104C | |
| Dge DnaB | Deinococcus geothermalis | Thermophilic, radiation |
| | DSM11300 | resistant |
| Dha-DCB2 RIR1 | Desulfitobacterium hafniense DCB-2 | Anaerobic dehalogenating |
| | | bacteria, taxon: 49338 |
| Dha-Y51 RIR1 | Desulfitobacterium hafniense Y51 | Anaerobic dehalogenating |
| | | bacteria, taxon: 138119 |
| Dpr-MLMSl RIR1 | delta proteobacterium MLMS-1 | Taxon: 262489 |
| Dra RIR1 | Deinococcus radiodurans R1, TIGR | Radiation resistant, |
| | strain | taxon: 1299 |
| Dra Snf2-c | Deinococcus radiodurans R1, TIGR | Radiation and DNA damage |
| | strain | resistent, taxon: 1299 |
| Dra Snf2-n | Deinococcus radiodurans R1, TIGR | Radiation and DNA damage |
| | strain | resistent, taxon: 1299 |
| Dra-ATCC13939 Snf2 | Deinococcus radiodurans R1, | Radiation and DNA damage |
| | ATCC13939/Brooks & Murray strain | resistent, taxon: 1299 |
| Dth UDP GD | Dictyoglomus thermophilum H-6-12 | strain = “H-6-12; ATCC 35947, |
| | | taxon: 309799 |
| Dvul ParB | Desulfovibrio vulgaris subsp. | taxon: 391774 |
| | vulgaris DP4 | |
| EP-Min27 Primase | Enterobacteria phage Min27 | bacteriphage of |
| | | host = “Escherichia coli |
| | | O157: H7 str. Min27” |
| Fal DnaB | Frankia alni ACN14a | Plant symbiot, taxon: 326424 |
| Fsp-CcI3 RIR1 | Frankia species CcI3 | taxon: 106370 |
| Gob DnaE | Gemmata obscuriglobus UQM2246 | Taxon 114, TIGR genome |
| | | strain, budding bacteria |
| Gob Hyp | Gemmata obscuriglobus UQM2246 | Taxon 114, TIGR genome |
| | | strain, budding bacteria |
| Gvi DnaB | Gloeobacter violaceus, PCC 7421 | taxon: 33072 |
| Gvi RIR1-1 | Gloeobacter violaceus, PCC 7421 | taxon: 33072 |
| Gvi RIR1-2 | Gloeobacter violaceus, PCC 7421 | taxon: 33072 |
| Hhal DnaB | Halorhodospira halophila SL1 | taxon: 349124 |
| Kfl-DSM17836 DnaB | Kribbella flavida DSM 17836 | Taxon: 479435 |
| Kra DnaB | Kineococcus radiotolerans | Radiation resistant |
| | SRS30216 | |
| LLP-KSY1 PolA | Lactococcus phage KSY1 | Bacteriophage, taxon: 388452 |
| LP-phiHSIC Helicase | Listonella pelagia phage phiHSIC | taxon: 310539, a |
| | | pseudotemperate marine |
| | | phage of Listonella pelagia |
| Lsp-PCC8106 GyrB | Lyngbya sp. PCC 8106 | Taxon: 313612 |
| MP-Be DnaB | Mycobacteriophage Bethlehem | Bacteriophage, taxon: 260121 |
| MP-Be gp51 | Mycobacteriophage Bethlehem | Bacteriophage, taxon: 260121 |
| MP-Catera gp206 | Mycobacteriophage Catera | Mycobacteriophage, |
| | | taxon: 373404 |
| MP-KBG gp53 | Mycobacterium phage KBG | Taxon: 540066 |
| MP-Mcjw1 DnaB | Mycobacteriophage CJW1 | Bacteriophage, taxon: 205869 |
| MP-Omega DnaB | Mycobacteriophage Omega | Bacteriophage, taxon: 205879 |
| MP-U2 gp50 | Mycobacteriophage U2 | Bacteriophage, taxon: 260120 |
| Maer-NIES843 DnaB | Microcystis aeruginosa NIES-843 | Bloom-forming toxic |
| | | cyanobacterium, taxon: 449447 |
| Maer-NIES843 DnaE-c | Microcystis aeruginosa NIES-843 | Bloom-forming toxic |
| | | cyanobacterium, taxon: 449447 |
| Maer-NIES843 DnaE-n | Microcystis aeruginosa NIES-843 | Bloom-forming toxic |
| | | cyanobacterium, taxon: 449447 |
| Mau-ATCC27029 | Micromonospora aurantiaca ATCC | Taxon: 644283 |
| GyrA | 27029 | |
| Mav-104 DnaB | Mycobacterium avium 104 | taxon: 243243 |
| Mav-ATCC25291 | Mycobacterium avium subsp. avium | Taxon: 553481 |
| DnaB | ATCC 25291 | |
| Mav-ATCC35712 | Mycobacterium avium | ATCC35712, taxon 1764 |
| DnaB | | |
| Mav-PT DnaB | Mycobacterium avium subsp. | taxon: 262316 |
| | paratuberculosis str. k10 | |
| Mbo Pps1 | Mycobacterium bovis subsp. bovis | strain = “AF2122/97”, |
| | AF2122/97 | taxon: 233413 |
| Mbo RecA | Mycobacterium bovis subsp. bovis | taxon: 233413 |
| | AF2122/97 | |
| Mbo SufB (Mbo Pps1) | Mycobacterium bovis subsp. bovis | taxon: 233413 |
| | AF2122/97 | |
| Mbo-1173P DnaB | Mycobacterium bovis BCG Pasteur | strain = BCG Pasteur |
| | 1173P | 1173P2,, taxon: 410289 |
| Mbo-AF2122 DnaB | Mycobacterium bovis subsp. bovis | strain = “AF2122/97”, |
| | AF2122/97 | taxon: 233413 |
| Mca MupF | Methylococcus capsulatus Bath, | prophage MuMc02, |
| | prophage MuMc02 | taxon: 243233 |
| Mca RIR1 | Methylococcus capsulatus Bath | taxon: 243233 |
| Mch RecA | Mycobacterium chitae | IP14116003, taxon: 1792 |
| Mcht-PCC7420 DnaE-1 | Microcoleus chthonoplastes | Cyanobacterium, |
| | PCC7420 | taxon: 118168 |
| Mcht-PCC7420 DnaE-2c | Microcoleus chthonoplastes | Cyanobacterium, |
| | PCC7420 | taxon: 118168 |
| Mcht-PCC7420 DnaE-2n | Microcoleus chthonoplastes | Cyanobacterium, |
| | PCC7420 | taxon: 118168 |
| Mcht-PCC7420 GyrB | Microcoleus chthonoplastes PCC | Taxon: 118168 |
| | 7420 | |
| Mcht-PCC7420 RIR1-1 | Microcoleus chthonoplastes PCC | Taxon: 118168 |
| | 7420 | |
| Mcht-PCC7420 RIR1-2 | Microcoleus chthonoplastes PCC | Taxon: 118168 |
| | 7420 | |
| Mex Helicase | Methylobacterium extorquens AM1 | Alphaproteobacteria |
| Mex TrbC | Methylobacterium extorquens AM1 | Alphaproteobacteria |
| Mfa RecA | Mycobacterium fallax | CITP8139, taxon: 1793 |
| Mfl GyrA | Mycobacterium flavescens Fla0 | taxon: 1776, reference |
| | | #930991 |
| Mfl RecA | Mycobacterium flavescens Fla0 | strain = Fla0, taxon: 1776, ref. |
| | | #930991 |
| Mfl-ATCC14474 RecA | Mycobacterium flavescens, | strain = ATCC14474, taxon: 1776, |
| | ATCC14474 | ref #930991 |
| Mfl-PYR-GCK DnaB | Mycobacterium flavescens PYR- | taxon: 350054 |
| | GCK | |
| Mga GyrA | Mycobacterium gastri | HP4389, taxon: 1777 |
| Mga RecA | Mycobacterium gastri | HP4389, taxon: 1777 |
| Mga SufB (Mga Pps1) | Mycobacterium gastri | HP4389, taxon: 1777 |
| Mgi-PYR-GCK DnaB | Mycobacterium gilvum PYR-GCK | taxon: 350054 |
| Mgi-PYR-GCK GyrA | Mycobacterium gilvum PYR-GCK | taxon: 350054 |
| Mgo GyrA | Mycobacterium gordonae | taxon: 1778, reference number |
| | | 930835 |
| Min-1442 DnaB | Mycobacterium intracellulare | strain 1442, taxon: 1767 |
| Min-ATCC13950 | Mycobacterium intracellulare ATCC | Taxon: 487521 |
| GyrA | 13950 | |
| Mkas GyrA | Mycobacterium kansasii | taxon: 1768 |
| Mkas-ATCC12478 | Mycobacterium kansasii ATCC | Taxon: 557599 |
| GyrA | 12478 | |
| Mle-Br4923 GyrA | Mycobacterium leprae Br4923 | Taxon: 561304 |
| Mle-TN DnaB | Mycobacterium leprae, strain TN | Human pathogen, taxon: 1769 |
| Mle-TN GyrA | Mycobacterium leprae TN | Human pathogen, |
| | | STRAIN = TN, taxon: 1769 |
| Mle-TN RecA | Mycobacterium leprae, strain TN | Human pathogen, taxon: 1769 |
| Mle-TN SufB (Mle | Mycobacterium leprae | Human pathogen, taxon: 1769 |
| Pps1) | | |
| Mma GyrA | Mycobacterium malmoense | taxon: 1780 |
| Mmag Magn8951 BIL | Magnetospirillum magnetotacticum | Gram negative, taxon: 272627 |
| | MS-1 | |
| Msh RecA | Mycobacterium shimodei | ATCC27962, taxon: 29313 |
| Msm DnaB-1 | Mycobacterium smegmatis MC2 155 | MC2 155, taxon: 246196 |
| Msm DnaB-2 | Mycobacterium smegmatis MC2 155 | MC2 155, taxon: 246196 |
| Msp-KMS DnaB | Mycobacterium species KMS | taxon: 189918 |
| Msp-KMS GyrA | Mycobacterium species KMS | taxon: 189918 |
| Msp-MCS DnaB | Mycobacterium species MCS | taxon: 164756 |
| Msp-MCS GyrA | Mycobacterium species MCS | taxon: 164756 |
| Mthe RecA | Mycobacterium thermoresistibile | ATCC19527, taxon: 1797 |
| Mtu SufB (Mtu Pps1) | Mycobacterium tuberculosis strains | Human pathogen, taxon: 83332 |
| | H37Rv & CDC1551 | |
| Mtu-C RecA | Mycobacterium tuberculosis C | Taxon: 348776 |
| Mtu-CDC1551 DnaB | Mycobacterium tuberculosis, | Human pathogen, taxon: 83332 |
| | CDC1551 | |
| Mtu-CPHL RecA | Mycobacterium tuberculosis | Taxon: 611303 |
| | CPHL_A | |
| Mtu-Canetti RecA | Mycobacterium tuberculosis/ | Taxon: 1773 |
| | strain = “Canetti” | |
| Mtu-EAS054 RecA | Mycobacterium tuberculosis EAS054 | Taxon: 520140 |
| Mtu-F11 DnaB | Mycobacterium tuberculosis, strain | taxon: 336982 |
| | F11 | |
| Mtu-H37Ra DnaB | Mycobacterium tuberculosis H37Ra | ATCC 25177, taxon: 419947 |
| Mtu-H37Rv DnaB | Mycobacterium tuberculosis H37Rv | Human pathogen, taxon: 83332 |
| Mtu-H37Rv RecA | Mycobacterium tuberculosis | Human pathogen, taxon: 83332 |
| | H37Rv, Also CDC1551 | |
| Mtu-Haarlem DnaB | Mycobacterium tuberculosis str. | Taxon: 395095 |
| | Haarlem | |
| Mtu-K85 RecA | Mycobacterium tuberculosis K85 | Taxon: 611304 |
| Mtu-R604 RecA-n | Mycobacterium tuberculosis ‘98- | Taxon: 555461 |
| | R604 INH-RIF-EM’ | |
| Mtu-So93 RecA | Mycobacterium tuberculosis | Human pathogen, taxon: 1773 |
| | So93/sub_species = “Canetti” | |
| Mtu-T17 RecA-c | Mycobacterium tuberculosis T17 | Taxon: 537210 |
| Mtu-T17 RecA-n | Mycobacterium tuberculosis T17 | Taxon: 537210 |
| Mtu-T46 RecA | Mycobacterium tuberculosis T46 | Taxon: 611302 |
| Mtu-T85 RecA | Mycobacterium tuberculosis T85 | Taxon: 520141 |
| Mtu-T92 RecA | Mycobacterium tuberculosis T92 | Taxon: 515617 |
| Mvan DnaB | Mycobacterium vanbaalenii PYR-1 | taxon: 350058 |
| Mvan GyrA | Mycobacterium vanbaalenii PYR-1 | taxon: 350058 |
| Mxa RAD25 | Myxococcus xanthus DK1622 | Deltaproteobacteria |
| Mxe GyrA | Mycobacterium xenopi strain | taxon: 1789 |
| | IMM5024 | |
| Naz-0708 RIR1-1 | Nostoc azollae 0708 | Taxon: 551115 |
| Naz-0708 RIR1-2 | Nostoc azollae 0708 | Taxon: 551115 |
| Nfa DnaB | Nocardia farcinica IFM 10152 | taxon: 247156 |
| Nfa Nfa15250 | Nocardia farcinica IFM 10152 | taxon: 247156 |
| Nfa RIR1 | Nocardia farcinica IFM 10152 | taxon: 247156 |
| Nosp-CCY9414 DnaE-n | Nodularia spumigena CCY9414 | Taxon: 313624 |
| Npu DnaB | Nostoc punctiforme | Cyanobacterium, taxon: 63737 |
| Npu GyrB | Nostoc punctiforme | Cyanobacterium, taxon: 63737 |
| Npu-PCC73102 DnaE-c | Nostoc punctiforme PCC73102 | Cyanobacterium, taxon: 63737, |
| | | ATCC29133 |
| Npu-PCC73102 DnaE-n | Nostoc punctiforme PCC73102 | Cyanobacterium, taxon: 63737, |
| | | ATCC29133 |
| Nsp-JS614 DnaB | Nocardioides species JS614 | taxon: 196162 |
| Nsp-JS614 TOPRIM | Nocardioides species JS614 | taxon: 196162 |
| Nsp-PCC7120 DnaB | Nostoc species PCC7120, (Anabaena | Cyanobacterium, Nitrogen- |
| | sp. PCC7120) | fixing, taxon: 103690 |
| Nsp-PCC7120 DnaE-c | Nostoc species PCC7120, (Anabaena | Cyanobacterium, Nitrogen- |
| | sp. PCC7120) | fixing, taxon: 103690 |
| Nsp-PCC7120 DnaE-n | Nostoc species PCC7120, (Anabaena | Cyanobacterium, Nitrogen- |
| | sp. PCC7120) | fixing, taxon: 103690 |
| Nsp-PCC7120 RIR1 | Nostoc species PCC7120, (Anabaena | Cyanobacterium, Nitrogen- |
| | sp. PCC7120) | fixing, taxon: 103690 |
| Oli DnaE-c | Oscillatoria limnetica str. ‘Solar Lake’ | Cyanobacterium, taxon: 262926 |
| Oli DnaE-n | Oscillatoria limnetica str. ‘Solar Lake’ | Cyanobacterium, taxon: 262926 |
| PP-PhiEL Helicase | Pseudomonas aeruginosa phage | Phage infects Pseudomonas |
| | phiEL | aeruginosa, taxon: 273133 |
| PP-PhiEL ORF11 | Pseudomonas aeruginosa phage | phage infects Pseudomonas |
| | phiEL | aeruginosa, taxon: 273133 |
| PP-PhiEL ORF39 | Pseudomonas aeruginosa phage | Phage infects Pseudomonas |
| | phiEL | aeruginosa, taxon: 273133 |
| PP-PhiEL ORF40 | Pseudomonas aeruginosa phage | phage infects Pseudomonas |
| | phiEL | aeruginosa, taxon: 273133 |
| Pfl Fha BIL | Pseudomonas fluorescens Pf-5 | Plant commensal organism, |
| | | taxon: 220664 |
| Plut RIR1 | Pelodictyon luteolum DSM 273 | Green sulfur bacteria, Taxon |
| | | 319225 |
| Pma-EXH1 GyrA | Persephonella marina EX-H1 | Taxon: 123214 |
| Pma-ExH1 DnaE | Persephonella marina EX-H1 | Taxon: 123214 |
| Pna RIR1 | Polaromonas naphthalenivorans CJ2 | taxon: 365044 |
| Pnuc DnaB | Polynucleobacter sp. QLW- | taxon: 312153 |
| | P1DMWA-1 | |
| Posp-JS666 DnaB | Polaromonas species JS666 | taxon: 296591 |
| Posp-JS666 RIR1 | Polaromonas species JS666 | taxon: 296591 |
| Pssp-A1-1 Fha | Pseudomonas species A1-1 | |
| Psy Fha | Pseudomonas syringae pv. tomato | Plant (tomato) pathogen, |
| | str. DC3000 | taxon: 223283 |
| Rbr-D9 GyrB | Raphidiopsis brookii D9 | Taxon: 533247 |
| Rce RIR1 | Rhodospirillum centenum SW | taxon: 414684, ATCC 51521 |
| Rer-SK121 DnaB | Rhodococcus erythropolis SK121 | Taxon: 596309 |
| Rma DnaB | Rhodothermus marinus | Thermophile, taxon: 29549 |
| Rma-DSM4252 DnaB | Rhodothermus marinus DSM 4252 | Taxon: 518766 |
| Rma-DSM4252 DnaE | Rhodothermus marinus DSM 4252 | Thermophile, taxon: 518766 |
| Rsp RIR1 | Roseovarius species 217 | taxon: 314264 |
| SaP-SETP12 dpol | Salmonella phage SETP12 | Phage, taxon: 424946 |
| SaP-SETP3 Helicase | Salmonella phage SETP3 | Phage, taxon: 424944 |
| SaP-SETP3 dpol | Salmonella phage SETP3 | Phage, taxon: 424944 |
| SaP-SETP5 dpol | Salmonella phage SETP5 | Phage, taxon: 424945 |
| Sare DnaB | Salinispora arenicola CNS-205 | taxon: 391037 |
| Sav RecG Helicase | Streptomyces avermitilis MA-4680 | taxon: 227882, ATCC 31267 |
| Sel-PC6301 RIR1 | Synechococcus elongatus PCC 6301 | taxon: 269084 Berkely strain |
| | | 6301~equivalent name: Ssp |
| | | PCC 6301~synonym: |
| | | Anacystis nudulans |
| Sel-PC7942 DnaE-c | Synechococcus elongatus PC7942 | taxon: 1140 |
| Sel-PC7942 DnaE-n | Synechococcus elongatus PC7942 | taxon: 1140 |
| Sel-PC7942 RIR1 | Synechococcus elongatus PC7942 | taxon: 1140 |
| Sel-PCC6301 DnaE-c | Synechococcus elongatus PCC 6301 | Cyanobacterium, |
| | and PCC7942 | taxon: 269084, “Berkely strain |
| | | 6301~equivalent name: |
| | | Synechococcus sp. PCC |
| | | 6301~synonym: Anacystis |
| | | nudulans” |
| Sel-PCC6301 DnaE-n | Synechococcus elongatus PCC 6301 | Cyanobacterium, |
| | | taxon: 269084“Berkely strain |
| | | 6301~equivalent name: |
| | | Synechococcus sp. PCC |
| | | 6301~synonym: Anacystis |
| | | nudulans” |
| Sep RIR1 | Staphylococcus epidermidis RP62A | taxon: 176279 |
| ShP-Sfv-2a-2457T-n | Shigella flexneri 2a str. 2457T | Putative bacteriphage |
| Primase | | |
| ShP-Sfv-2a-301-n | Shigella flexneri 2a str. 301 | Putative bacteriphage |
| Primase | | |
| ShP-Sfv-5 Primase | Shigella flexneri 5 str. 8401 | Bacteriphage, isolation_source_epidemic, |
| | | taxon: 373384 |
| SoP-SO1 dpol | Sodalis phage SO-1 | Phage/isolation_source = “Sodalis |
| | | glossinidius strain GA-SG, |
| | | secondary symbiont of |
| | | Glossina austeni (Newstead)” |
| Spl DnaX | Spirulina platensis, strain C1 | Cyanobacterium, taxon: 1156 |
| Sru DnaB | Salinibacter ruber DSM 13855 | taxon: 309807, strain = “DSM |
| | | 13855; M31” |
| Sru PolBc | Salinibacter ruber DSM 13855 | taxon: 309807, strain = “DSM |
| | | 13855; M31” |
| Sru RIR1 | Salinibacter ruber DSM 13855 | taxon: 309807, strain = “DSM |
| | | 13855; M31” |
| Ssp DnaB | Synechocystis species, strain | Cyanobacterium, taxon: 1148 |
| | PCC6803 | |
| Ssp DnaE-c | Synechocystis species, strain | Cyanobacterium, taxon: 1148 |
| | PCC6803 | |
| Ssp DnaE-n | Synechocystis species, strain | Cyanobacterium, taxon: 1148 |
| | PCC6803 | |
| Ssp DnaX | Synechocystis species, strain | Cyanobacterium, taxon: 1148 |
| | PCC6803 | |
| Ssp GyrB | Synechocystis species, strain | Cyanobacterium, taxon: 1148 |
| | PCC6803 | |
| Ssp-JA2 DnaB | Synechococcus species JA-2-3B′a(2-13) | Cyanobacterium, Taxon: 321332 |
| Ssp-JA2 RIR1 | Synechococcus species JA-2-3B′a(2-13) | Cyanobacterium, Taxon: 321332 |
| Ssp-JA3 DnaB | Synechococcus species JA-3-3Ab | Cyanobacterium, Taxon: 321327 |
| Ssp-JA3 RIR1 | Synechococcus species JA-3-3Ab | Cyanobacterium, Taxon: 321327 |
| Ssp-PCC7002 DnaE-c | Synechocystis species, strain PCC | Cyanobacterium, taxon: 32049 |
| | 7002 | |
| Ssp-PCC7002 DnaE-n | Synechocystis species, strain PCC | Cyanobacterium, taxon: 32049 |
| | 7002 | |
| Ssp-PCC7335 RIR1 | Synechococcus sp. PCC 7335 | Taxon: 91464 |
| StP-Twort ORF6 | Staphylococcus phage Twort | Phage, taxon 55510 |
| Susp-NBC371 DnaB | Sulfurovum sp. NBC37-1 | taxon: 387093 |
| intein | | |
| Taq-Y51MC23 DnaE | Thermus aquaticus Y51MC23 | Taxon: 498848 |
| Taq-Y51MC23 RIR1 | Thermus aquaticus Y51MC23 | Taxon: 498848 |
| Tcu-DSM43183 RecA | Thermomonospora curvata DSM | Taxon: 471852 |
| | 43183 | |
| Tel DnaE-c | Thermosynechococcus elongatus BP-1 | Cyanobacterium, taxon: 197221 |
| Tel DnaE-n | Thermosynechococcus elongatus BP-1 | Cyanobacterium, |
| Ter DnaB-1 | Trichodesmium erythraeum IMS101 | Cyanobacterium, taxon: 203124 |
| Ter DnaB-2 | Trichodesmium erythraeum IMS101 | Cyanobacterium, taxon: 203124 |
| Ter DnaE-1 | Trichodesmium erythraeum IMS101 | Cyanobacterium, taxon: 203124 |
| Ter DnaE-2 | Trichodesmium erythraeum IMS101 | Cyanobacterium, taxon: 203124 |
| Ter DnaE-3c | Trichodesmium erythraeum IMS101 | Cyanobacterium, taxon: 203124 |
| Ter DnaE-3n | Trichodesmium erythraeum IMS101 | Cyanobacterium, taxon: 203124 |
| Ter GyrB | Trichodesmium erythraeum IMS101 | Cyanobacterium, taxon: 203124 |
| Ter Ndse-1 | Trichodesmium erythraeum IMS101 | Cyanobacterium, taxon: 203124 |
| Ter Ndse-2 | Trichodesmium erythraeum IMS101 | Cyanobacterium, taxon: 203124 |
| Ter RIR1-1 | Trichodesmium erythraeum IMS101 | Cyanobacterium, taxon: 203124 |
| Ter RIR1-2 | Trichodesmium erythraeum IMS101 | Cyanobacterium, taxon: 203124 |
| Ter RIR1-3 | Trichodesmium erythraeum IMS101 | Cyanobacterium, taxon: 203124 |
| Ter RIR1-4 | Trichodesmium erythraeum IMS101 | Cyanobacterium, taxon: 203124 |
| Ter Snf2 | Trichodesmium erythraeum IMS101 | Cyanobacterium, taxon: 203124 |
| Ter ThyX | Trichodesmium erythraeum IMS101 | Cyanobacterium, taxon: 203124 |
| Tfus RecA-1 | Thermobifida fusca YX | Thermophile, taxon: 269800 |
| Tfus RecA-2 | Thermobifida fusca YX | Thermophile, taxon: 269800 |
| Tfus Tfu2914 | Thermobifida fusca YX | Thermophile, taxon: 269800 |
| Thsp-K90 RIR1 | Thioalkalivibrio sp. K90mix | Taxon: 396595 |
| Tth-DSM571 RIR1 | Thermoanaerobacterium | Taxon: 580327 |
| | thermosaccharolyticum DSM 571 | |
| Tth-HB27 DnaE-1 | Thermus thermophilus HB27 | thermophile, taxon: 262724 |
| Tth-HB27 DnaE-2 | Thermus thermophilus HB27 | thermophile, taxon: 262724 |
| Tth-HB27 RIR1-1 | Thermus thermophilus HB27 | thermophile, taxon: 262724 |
| Tth-HB27 RIR1-2 | Thermus thermophilus HB27 | thermophile, taxon: 262724 |
| Tth-HB8 DnaE-1 | Thermus thermophilus HB8 | thermophile, taxon: 300852 |
| Tth-HB8 DnaE-2 | Thermus thermophilus HB8 | thermophile, taxon: 300852 |
| Tth-HB8 RIR1-1 | Thermus thermophilus HB8 | thermophile, taxon: 300852 |
| Tth-HB8 RIR1-2 | Thermus thermophilus HB8 | thermophile, taxon: 300852 |
| Tvu DnaE-c | Thermosynechococcus vulcanus | Cyanobacterium, taxon: 32053 |
| Tvu DnaE-n | Thermosynechococcus vulcanus | Cyanobacterium, taxon: 32053 |
| Tye RNR-1 | Thermodesulfovibrio yellowstonii | taxon: 289376 |
| | DSM 11347 | |
| Tye RNR-2 | Thermodesulfovibrio yellowstonii | taxon: 289376 |
| | DSM 11347 | |
| Archaea | | |
| Ape APE0745 | Aeropyrum pernix K1 | Thermophile, taxon: 56636 |
| Cme-boo Pol-II | Candidatus Methanoregula boonei | taxon: 456442 |
| | 6A8 | |
| Fac-Fer1 RIR1 | Ferroplasma acidarmanus, | strain Fer1, eats iron |
| | taxon: 97393 and taxon 261390 | |
| Fac-Fer1 SufB (Fac | Ferroplasma acidarmanus | strain fer1, eats |
| Pps1) | | iron, taxon: 97393 |
| Fac-TypeI RIR1 | Ferroplasma acidarmanus type I, | Eats iron, taxon 261390 |
| Fac-typeI SufB (Fac | Ferroplasma acidarmanus | Eats iron, taxon: 261390 |
| Pps1) | | |
| Hma CDC21 | Haloarcula marismortui ATCC | taxon: 272569, |
| | 43049 | |
| Hma Pol-II | Haloarcula marismortui ATCC | taxon: 272569, |
| | 43049 | |
| Hma PolB | Haloarcula marismortui ATCC | taxon: 272569, |
| | 43049 | |
| Hma TopA | Haloarcula marismortui ATCC | taxon: 272569 |
| | 43049 | |
| Hmu-DSM12286 | Halomicrobium mukohataei DSM | taxon: 485914 (Halobacteria) |
| MCM | 12286 | |
| Hmu-DSM12286 PolB | Halomicrobium mukohataei DSM | Taxon: 485914 |
| | 12286 | |
| Hsa-R1 MCM | Halobacterium salinarum R-1 | Halophile, |
| | | taxon: 478009, strain = “R1; |
| | | DSM 671” |
| Hsp-NRC1 CDC21 | Halobacterium species NRC-1 | Halophile, taxon: 64091 |
| Hsp-NRC1 Pol-II | Halobacterium salinarum NRC-1 | Halophile, taxon: 64091 |
| Hut MCM-2 | Halorhabdus utahensis DSM 12940 | taxon: 519442 |
| Hut-DSM12940 MCM-1 | Halorhabdus utahensis DSM 12940 | taxon: 519442 |
| Hvo PolB | Haloferax volcanii DS70 | taxon: 2246 |
| Hwa GyrB | Haloquadratum walsbyi DSM 16790 | Halophile, taxon: 362976, |
| | | strain: DSM 16790 = |
| | | HBSQ001 |
| Hwa MCM-1 | Haloquadratum walsbyi DSM 16790 | Halophile, taxon: 362976, |
| | | strain: DSM 16790 = |
| | | HBSQ001 |
| Hwa MCM-2 | Haloquadratum walsbyi DSM 16790 | Halophile, taxon: 362976, |
| | | strain: DSM 16790 = |
| | | HBSQ001 |
| Hwa MCM-3 | Haloquadratum walsbyi DSM 16790 | Halophile, taxon: 362976, |
| | | strain: DSM 16790 = |
| | | HBSQ001 |
| Hwa MCM-4 | Haloquadratum walsbyi DSM 16790 | Halophile, taxon: 362976, |
| | | strain: DSM 16790 = |
| | | HBSQ001 |
| Hwa Pol-II-1 | Haloquadratum walsbyi DSM 16790 | Halophile, taxon: 362976, |
| | | strain: DSM 16790 = |
| | | HBSQ001 |
| Hwa Pol-II-2 | Haloquadratum walsbyi DSM 16790 | Halophile, taxon: 362976, |
| | | strain: DSM 16790 = |
| | | HBSQ001 |
| Hwa PolB-1 | Haloquadratum walsbyi DSM 16790 | Halophile, taxon: 362976, |
| | | strain: DSM 16790 = |
| | | HBSQ001 |
| Hwa PolB-2 | Haloquadratum walsbyi DSM 16790 | Halophile, taxon: 362976, |
| | | strain: DSM 16790 = |
| | | HBSQ001 |
| Hwa PolB-3 | Haloquadratum walsbyi DSM 16790 | Halophile, taxon: 362976, |
| | | strain: DSM 16790 = |
| | | HBSQ001 |
| Hwa RCF | Haloquadratum walsbyi DSM 16790 | Halophile, taxon: 362976, |
| | | strain: DSM 16790 = |
| | | HBSQ001 |
| Hwa RIR1-1 | Haloquadratum walsbyi DSM 16790 | Halophile, taxon: 362976, |
| | | strain: DSM 16790 = |
| | | HBSQ001 |
| Hwa RIR1-2 | Haloquadratum walsbyi DSM 16790 | Halophile, taxon: 362976, |
| | | strain: DSM 16790 = |
| | | HBSQ001 |
| Hwa Top6B | Haloquadratum walsbyi DSM 16790 | Halophile, taxon: 362976, |
| | | strain: DSM 16790 = |
| | | HBSQ001 |
| Hwa rPol A″ | Haloquadratum walsbyi DSM 16790 | Halophile, taxon: 362976, |
| | | strain: DSM 16790 = |
| | | HBSQ001 |
| Maeo Pol-II | Methanococcus aeolicus Nankai-3 | taxon: 419665 |
| Maeo RFC | Methanococcus aeolicus Nankai-3 | taxon: 419665 |
| Maeo RNR | Methanococcus aeolicus Nankai-3 | taxon: 419665 |
| Maeo-N3 Helicase | Methanococcus aeolicus Nankai-3 | taxon: 419665 |
| Maeo-N3 RtcB | Methanococcus aeolicus Nankai-3 | taxon: 419665 |
| Maeo-N3 UDP GD | Methanococcus aeolicus Nankai-3 | taxon: 419665 |
| Mein-ME PEP | Methanocaldococcus infernus ME | thermophile, Taxon: 573063 |
| Mein-ME RFC | Methanocaldococcus infernus ME | Taxon: 573063 |
| Memar MCM2 | Methanoculleus marisnigri JR1 | taxon: 368407 |
| Memar Pol-II | Methanoculleus marisnigri JR1 | taxon: 368407 |
| Mesp-FS406 PolB-1 | Methanocaldococcus sp. FS406-22 | Taxon: 644281 |
| Mesp-FS406 PolB-2 | Methanocaldococcus sp. FS406-22 | Taxon: 644281 |
| Mesp-FS406 PolB-3 | Methanocaldococcus sp. FS406-22 | Taxon: 644281 |
| Mesp-FS406-22 LHR | Methanocaldococcus sp. FS406-22 | Taxon: 644281 |
| Mfe-AG86 Pol-1 | Methanocaldococcus fervens AG86 | Taxon: 573064 |
| Mfe-AG86 Pol-2 | Methanocaldococcus fervens AG86 | Taxon: 573064 |
| Mhu Pol-II | Methanospirillum hungateii JF-1 | taxon 323259 |
| Mja GF-6P | Methanococcus jannaschii | Thermophile, DSM 2661, |
| | (Methanocaldococcus jannaschii | taxon: 2190 |
| | DSM 2661) | |
| Mja Helicase | Methanococcus jannaschii | Thermophile, DSM 2661, |
| | (Methanocaldococcus jannaschii | taxon: 2190 |
| | DSM 2661) | |
| Mja Hyp-1 | Methanococcus jannaschii | Thermophile, DSM 2661, |
| | (Methanocaldococcus jannaschii | taxon: 2190 |
| | DSM 2661) | |
| Mja IF2 | Methanococcus jannaschii | Thermophile, DSM 2661, |
| | (Methanocaldococcus jannaschii | taxon: 2190 |
| | DSM 2661) | |
| Mja KlbA | Methanococcus jannaschii | Thermophile, DSM 2661, |
| | (Methanocaldococcus jannaschii | taxon: 2190 |
| | DSM 2661) | |
| Mja PEP | Methanococcus jannaschii | Thermophile, DSM 2661, |
| | (Methanocaldococcus jannaschii | taxon: 2190 |
| | DSM 2661) | |
| Mja Pol-1 | Methanococcus jannaschii | Thermophile, DSM 2661, |
| | (Methanocaldococcus jannaschii | taxon: 2190 |
| | DSM 2661) | |
| Mja Pol-2 | Methanococcus jannaschii | Thermophile, DSM 2661, |
| | (Methanocaldococcus jannaschii | taxon: 2190 |
| | DSM 2661) | |
| Mja RFC-1 | Methanococcus jannaschii | Thermophile, DSM 2661, |
| | (Methanocaldococcus jannaschii | taxon: 2190 |
| | DSM 2661) | |
| Mja RFC-2 | Methanococcus jannaschii | Thermophile, DSM 2661, |
| | (Methanocaldococcus jannaschii | taxon: 2190 |
| | DSM 2661) | |
| Mja RFC-3 | Methanococcus jannaschii | Thermophile, DSM 2661, |
| | (Methanocaldococcus jannaschii | taxon: 2190 |
| | DSM 2661) | |
| Mja RNR-1 | Methanococcus jannaschii | Thermophile, DSM 2661, |
| | (Methanocaldococcus jannaschii | taxon: 2190 |
| | DSM 2661) | |
| Mja RNR-2 | Methanococcus jannaschii | Thermophile, DSM 2661, |
| | (Methanocaldococcus jannaschii | taxon: 2190 |
| | DSM 2661) | |
| Mja RtcB (Mja Hyp-2) | Methanococcus jannaschii | Thermophile, DSM 2661, |
| | (Methanocaldococcus jannaschii | taxon: 2190 |
| | DSM 2661) | |
| Mja TFIIB | Methanococcus jannaschii | Thermophile, DSM 2661, |
| | (Methanocaldococcus jannaschii | taxon: 2190 |
| | DSM 2661) | |
| Mja UDP GD | Methanococcus jannaschii | Thermophile, DSM 2661, |
| | (Methanocaldococcus jannaschii | taxon: 2190 |
| | DSM 2661) | |
| Mja r-Gyr | Methanococcus jannaschii | Thermophile, DSM 2661, |
| | (Methanocaldococcus jannaschii | taxon: 2190 |
| | DSM 2661) | |
| Mja rPol A′ | Methanococcus jannaschii | Thermophile, DSM 2661, |
| | (Methanocaldococcus jannaschii | taxon: 2190 |
| | DSM 2661) | |
| Mja rPol A″ | Methanococcus jannaschii | Thermophile, DSM 2661, |
| | (Methanocaldococcus jannaschii | taxon: 2190 |
| | DSM 2661) | |
| Mka CDC48 | Methanopyrus kandleri AV19 | Thermophile, taxon: 190192 |
| Mka EF2 | Methanopyrus kandleri AV19 | Thermophile, taxon: 190192 |
| Mka RFC | Methanopyrus kandleri AV19 | Thermophile, taxon: 190192 |
| Mka RtcB | Methanopyrus kandleri AV19 | Thermophile, taxon: 190192 |
| Mka VatB | Methanopyrus kandleri AV19 | Thermophile, taxon: 190192 |
| Mth RIR1 | Methanothermobacter | Thermophile, delta H strain |
| | thermautotrophicus | |
| | (Methanobacterium | |
| | thermoautotrophicum) | |
| Mvu-M7 Helicase | Methanocaldococcus vulcanius M7 | Taxon: 579137 |
| Mvu-M7 Pol-1 | Methanocaldococcus vulcanius M7 | Taxon: 579137 |
| Mvu-M7 Pol-2 | Methanocaldococcus vulcanius M7 | Taxon: 579137 |
| Mvu-M7 Pol-3 | Methanocaldococcus vulcanius M7 | Taxon: 579137 |
| Mvu-M7 UDP GD | Methanocaldococcus vulcanius M7 | Taxon: 579137 |
| Neq Pol-c | Nanoarchaeum equitans Kin4-M | Thermophile, taxon: 228908 |
| Neq Pol-n | Nanoarchaeum equitans Kin4-M | Thermophile, taxon: 228908 |
| Nma-ATCC43099 | Natrialba magadii ATCC 43099 | Taxon: 547559 |
| MCM | | |
| Nma-ATCC43099 | Natrialba magadii ATCC 43099 | Taxon: 547559 |
| PolB-1 | | |
| Nma-ATCC43099 | Natrialba magadii ATCC 43099 | Taxon: 547559 |
| PolB-2 | | |
| Nph CDC21 | Natronomonas pharaonis DSM 2160 | taxon: 348780 |
| Nph PolB-1 | Natronomonas pharaonis DSM 2160 | taxon: 348780 |
| Nph PolB-2 | Natronomonas pharaonis DSM 2160 | taxon: 348780 |
| Nph rPol A″ | Natronomonas pharaonis DSM 2160 | taxon: 348780 |
| Pab CDC21-1 | Pyrococcus abyssi | Thermophile, strain Orsay, |
| | | taxon: 29292 |
| Pab CDC21-2 | Pyrococcus abyssi | Thermophile, strain Orsay, |
| | | taxon: 29292 |
| Pab IF2 | Pyrococcus abyssi | Thermophile, strain Orsay, |
| | | taxon: 29292 |
| Pab KlbA | Pyrococcus abyssi | Thermophile, strain Orsay, |
| | | taxon: 29292 |
| Pab Lon | Pyrococcus abyssi | Thermophile, strain Orsay, |
| | | taxon: 29292 |
| Pab Moaa | Pyrococcus abyssi | Thermophile, strain Orsay, |
| | | taxon: 29292 |
| Pab Pol-II | Pyrococcus abyssi | Thermophile, strain Orsay, |
| | | taxon: 29292 |
| Pab RFC-1 | Pyrococcus abyssi | Thermophile, strain Orsay, |
| | | taxon: 29292 |
| Pab RFC-2 | Pyrococcus abyssi | Thermophile, strain Orsay, |
| | | taxon: 29292 |
| Pab RIR1-1 | Pyrococcus abyssi | Thermophile, strain Orsay, |
| | | taxon: 29292 |
| Pab RIR1-2 | Pyrococcus abyssi | Thermophile, strain Orsay, |
| | | taxon: 29292 |
| Pab RIR1-3 | Pyrococcus abyssi | Thermophile, strain Orsay, |
| | | taxon: 29292 |
| Pab RtcB (Pab Hyp-2) | Pyrococcus abyssi | Thermophile, strain Orsay, |
| | | taxon: 29292 |
| Pab VMA | Pyrococcus abyssi | Thermophile, strain Orsay, |
| | | taxon: 29292 |
| Par RIR1 | Pyrobaculum arsenaticum DSM | taxon: 340102 |
| | 13514 | |
| Pfu CDC21 | Pyrococcus furiosus | Thermophile, taxon: 186497, |
| | | DSM3638 |
| Pfu IF2 | Pyrococcus furiosus | Thermophile, taxon: 186497, |
| | | DSM3638 |
| Pfu KlbA | Pyrococcus furiosus | Thermophile, taxon: 186497, |
| | | DSM3638 |
| Pfu Lon | Pyrococcus furiosus | Thermophile, taxon: 186497, |
| | | DSM3638 |
| Pfu RFC | Pyrococcus furiosus | Thermophile, DSM3638, |
| | | taxon: 186497 |
| Pfu RIR1-1 | Pyrococcus furiosus | Thermophile, taxon: 186497, |
| | | DSM3638 |
| Pfu RIR1-2 | Pyrococcus furiosus | Thermophile, taxon: 186497, |
| | | DSM3638 |
| Pfu RtcB (Pfu Hyp-2) | Pyrococcus furiosus | Thermophile, taxon: 186497, |
| | | DSM3638 |
| Pfu TopA | Pyrococcus furiosus | Thermophile, taxon: 186497, |
| | | DSM3638 |
| Pfu VMA | Pyrococcus furiosus | Thermophile, taxon: 186497, |
| | | DSM3638 |
| Pho CDC21-1 | Pyrococcus horikoshii OT3 | Thermophile, taxon: 53953 |
| Pho CDC21-2 | Pyrococcus horikoshii OT3 | Thermophile, taxon: 53953 |
| Pho IF2 | Pyrococcus horikoshii OT3 | Thermophile, taxon: 53953 |
| Pho KlbA | Pyrococcus horikoshii OT3 | Thermophile, taxon: 53953 |
| Pho LHR | Pyrococcus horikoshii OT3 | Thermophile, taxon: 53953 |
| Pho Lon | Pyrococcus horikoshii OT3 | Thermophile, taxon: 53953 |
| Pho Pol I | Pyrococcus horikoshii OT3 | Thermophile, taxon: 53953 |
| Pho Pol-II | Pyrococcus horikoshii OT3 | Thermophile, taxon: 53953 |
| Pho RFC | Pyrococcus horikoshii OT3 | Thermophile, taxon: 53953 |
| Pho RIR1 | Pyrococcus horikoshii OT3 | Thermophile, taxon: 53953 |
| Pho RadA | Pyrococcus horikoshii OT3 | Thermophile, taxon: 53953 |
| Pho RtcB (Pho Hyp-2) | Pyrococcus horikoshii OT3 | Thermophile, taxon: 53953 |
| Pho VMA | Pyrococcus horikoshii OT3 | Thermophile, taxon: 53953 |
| Pho r-Gyr | Pyrococcus horikoshii OT3 | Thermophile, taxon: 53953 |
| Psp-GBD Pol | Pyrococcus species GB-D | Thermophile |
| Pto VMA | Picrophilus torridus, DSM 9790 | DSM 9790, taxon: 263820, |
| | | Thermoacidophile |
| Smar 1471 | Staphylothermus marinus F1 | taxon: 399550 |
| Smar MCM2 | Staphylothermus marinus F1 | taxon: 399550 |
| Tac-ATCC25905 VMA | Thermoplasma acidophilum, ATCC | Thermophile, taxon: 2303 |
| | 25905 | |
| Tac-DSM1728 VMA | Thermoplasma acidophilum, | Thermophile, taxon: 2303 |
| | DSM1728 | |
| Tag Pol-1 (Tsp-TY Pol-1) | Thermococcus aggregans | Thermophile, taxon: 110163 |
| Tag Pol-2 (Tsp-TY Pol-2) | Thermococcus aggregans | Thermophile, taxon: 110163 |
| Tag Pol-3 (Tsp-TY Pol-3) | Thermococcus aggregans | Thermophile, taxon: 110163 |
| Tba Pol-II | Thermococcus barophilus MP | taxon: 391623 |
| Tfu Pol-1 | Thermococcus fumicolans | Thermophilem, taxon: 46540 |
| Tfu Pol-2 | Thermococcus fumicolans | Thermophile, taxon: 46540 |
| Thy Pol-1 | Thermococcus hydrothermalis | Thermophile, taxon: 46539 |
| Thy Pol-2 | Thermococcus hydrothermalis | Thermophile, taxon: 46539 |
| Tko CDC21-1 | Thermococcus kodakaraensis KOD1 | Thermophile, taxon: 69014 |
| Tko CDC21-2 | Thermococcus kodakaraensis KOD1 | Thermophile, taxon: 69014 |
| Tko Helicase | Thermococcus kodakaraensis KOD1 | Thermophile, taxon: 69014 |
| Tko IF2 | Thermococcus kodakaraensis KOD1 | Thermophile, taxon: 69014 |
| Tko KlbA | Thermococcus kodakaraensis KOD1 | Thermophile, taxon: 69014 |
| Tko LHR | Thermococcus kodakaraensis KOD1 | Thermophile, taxon: 69014 |
| Tko Pol-1 (Pko Pol-1) | Pyrococcus/Thermococcus | Thermophile, taxon: 69014 |
| | kodakaraensis KOD1 | |
| Tko Pol-2 (Pko Pol-2) | Pyrococcus/Thermococcus | Thermophile, taxon: 69014 |
| | kodakaraensis KOD1 | |
| Tko Pol-II | Thermococcus kodakaraensis KOD1 | Thermophile, taxon: 69014 |
| Tko RFC | Thermococcus kodakaraensis KOD1 | Thermophile, taxon: 69014 |
| Tko RIR1-1 | Thermococcus kodakaraensis KOD1 | Thermophile, taxon: 69014 |
| Tko RIR1-2 | Thermococcus kodakaraensis KOD1 | Thermophile, taxon: 69014 |
| Tko RadA | Thermococcus kodakaraensis KOD1 | Thermophile, taxon: 69014 |
| Tko TopA | Thermococcus kodakaraensis KOD1 | Thermophile, taxon: 69014 |
| Tko r-Gyr | Thermococcus kodakaraensis KOD1 | Thermophile, taxon: 69014 |
| Tli Pol-1 | Thermococcus litoralis | Thermophile, taxon: 2265 |
| Tli Pol-2 | Thermococcus litoralis | Thermophile, taxon: 2265 |
| Tma Pol | Thermococcus marinus | taxon: 187879 |
| Ton-NA1 LHR | Thermococcus onnurineus NA1 | Taxon: 523850 |
| Ton-NA1 Pol | Thermococcus onnurineus NA1 | taxon: 342948 |
| Tpe Pol | Thermococcus peptonophilus strain | taxon: 32644 |
| | SM2 | |
| Tsi-MM739 Lon | Thermococcus sibiricus MM 739 | Thermophile, Taxon: 604354 |
| Tsi-MM739 Pol-1 | Thermococcus sibiricus MM 739 | Taxon: 604354 |
| Tsi-MM739 Pol-2 | Thermococcus sibiricus MM 739 | Taxon: 604354 |
| Tsi-MM739 RFC | Thermococcus sibiricus MM 739 | Taxon: 604354 |
| Tsp AM4 RtcB | Thermococcus sp. AM4 | Taxon: 246969 |
| Tsp-AM4 LHR | Thermococcus sp. AM4 | Taxon: 246969 |
| Tsp-AM4 Lon | Thermococcus sp. AM4 | Taxon: 246969 |
| Tsp-AM4 RIR1 | Thermococcus sp. AM4 | Taxon: 246969 |
| Tsp-GE8 Pol-1 | Thermococcus species GE8 | Thermophile, taxon: 105583 |
| Tsp-GE8 Pol-2 | Thermococcus species GE8 | Thermophile, taxon: 105583 |
| Tsp-GT Pol-1 | Thermococcus species GT | taxon: 370106 |
| Tsp-GT Pol-2 | Thermococcus species GT | taxon: 370106 |
| Tsp-OGL-20P Pol | Thermococcus sp. OGL-20P | taxon: 277988 |
| Tthi Pol | Thermococcus thioreducens | Hyperthermophile |
| Tvo VMA | Thermoplasma volcanium GSS1 | Thermophile, taxon: 50339 |
| Tzi Pol | Thermococcus zilligii | taxon: 54076 |
| Unc-ERS PFL | uncultured archaeon Gzfos13E1 | isolation_source = “Eel River |
| | | sediment”, |
| | | clone = “GZfos13E1”, |
| | | taxon: 285397 |
| Unc-ERS RIR1 | uncultured archaeon GZfos9C4 | isolation_source = “Eel River |
| | | sediment”, taxon: 285366, |
| | | clone = “GZfos9C4” |
| Unc-ERS RNR | uncultured archaeon GZfos10C7 | isolation_source = “Eel River |
| | | sediment”, |
| | | clone = “GZfos10C7”, |
| | | taxon: 285400 |
| Unc-MetRFS MCM2 | uncultured archaeon (Rice Cluster I) | Enriched methanogenic |
| | | consortium from rice field |
| | | soil, taxon: 198240 |

The split inteins of the disclosed compositions or that can be used in the disclosed methods can be modified, or mutated, inteins. A modified intein can comprise modifications to the N-terminal intein segment, the C-terminal intein segment, or both. The modifications can include additional amino acids at the N-terminus the C-terminus of either portion of the split intein, or can be within the either portion of the split intein. Table 2 shows a list of amino acids, their abbreviations, polarity, and charge.

TABLE 2
List of Amino Acids

| | 3-Letter | 1-Letter | | |
|---|---|---|---|---|
| Amino Acid | Code | Code | Polarity | Charge |
| Alanine | Ala | A | nonpolar | neutral |
| Arginine | Arg | R | Basic | positive |
| | | | polar | |
| Asparagine | Asn | N | polar | neutral |
| Aspartic acid | Asp | D | acidic negative | |
| | | | polar | |
| Cysteine | Cys | C | nonpolar | neutral |
| Glutamic acid | Glu | E | acidic | negative |
| | | | polar | |
| Glutamine | Gln | Q | polar | neutral |
| Glycine | Gly | G | nonpolar | neutral |
| Histidine | His | H | Basic | Positive (10%) |
| | | | polar | Neutral (90%) |
| Isoleucine | Ile | I | nonpolar | neutral |
| Leucine | Leu | L | nonpolar | neutral |
| Lysine | Lys | K | Basic | positive |
| | | | polar | |
| Methionine | Met | M | nonpolar | neutral |
| Phenylalanine | Phe | F | nonpolar | neutral |
| Proline | Pro | P | nonpolar | neutral |
| Serine | Ser | S | polar | neutral |
| Threonine | Thr | T | polar | neutral |
| Tryptophan | Trp | W | nonpolar | neutral |
| Tyrosine | Tyr | Y | polar | neutral |
| Valine | Val | V | nonpolar | neutral |

The N-intein of the invention may be coupled to solid phase, such as a membrane, fiber, particle, bead or chip. The solid phase may be a chromatography resin of natural or synthetic origin, such as a natural or synthetic resin, preferably a polysaccharide such as agarose. The solid phase, such as a chromatography resin, may be provided with embedded magnetic particles. In another embodiment the solid phase is a non-diffusion limited resin/fibrous material.

In this case the solid phase may be formed from one or more polymeric nanofibre substrates, such as electrospun polymer nanofibres. Polymer nanofibres for use in the present invention typically have mean diameters from 10 nm to 1000 nm. The length of polymer nanofibres is not particularly limited. The polymer nanofibres can suitably be monofilament nanofibres and may e.g. have a circular, ellipsoidal or essentially circular/ellipsoidal cross section. Typically, the one or more polymer nanofibres are provided in the form of one or more non-woven sheets, each comprising one or more polymer nanofibers. A non-woven sheet comprising one or more polymer nanofibres is a mat of said one or more polymer nanofibres with each nanofibre oriented essentially randomly, i.e. it has not been fabricated so that the nanofibre or nanofibres adopts a particular pattern. Non-woven sheets typically have area densities from 1 to 40 g/m2. Non-woven sheets typically have a thickness from 5 to 120 μm. The polymer should be a polymer suitable for use as a chromatography medium, i.e. an adsorbent, in a chromatography method. Suitable polymers include polyamides such as nylon, polyacrylic acid, polymethacrylic acid, polyacrylonitrile, polystyrene, polysulfones e.g. polyethersulfone (PES), polycaprolactone, collagen, chitosan, polyethylene oxide, agarose, agarose acetate, cellulose, cellulose acetate, and combinations thereof.

The N-intein according to the invention may be immobilized on a solid support in a very high degree, 0.2-2 μmole/ml N-intein is coupled per ml resin (swollen gel).

The N-intein according to the invention may be coupled to the solid phase via a Lys-tail, comprising one or more Lys, such as at least two, on the C-terminal. Alternatively, the N-intein is coupled to the solid phase via a Cys-tail on the C-terminal.

### C-Intein Protein Variants

Preferably the invention also provides a C-intein comprising a split intein C-intein sequence or engineered variants thereof.

It will be appreciated that selection of the N-intein and C-intein can be from the same wild type split intein (e.g., both from Npu, or a variant of either the N- or C-intein, or alternatively can be selected from different wild type split inteins or the consensus split intein sequences, as it has been discovered that the affinity of a N-fragment for a different C-fragment (e.g., Npu N-fragment or variant thereof with Ssp C-fragment or variant thereof) still maintains sufficient binding affinity for use in the disclosed methods.

### Split Intein Systems

Preferably, the invention provides a split intein system for affinity purification of a protein of interest (POI), comprising a N-intein and C-intein as described above.

Preferably the N-intein is attached to a solid phase and the C-intein is co-expressed with the POI and used as a tag for affinity purification of the POI. Vice versa is also possible, ie attaching the C-intein to a solid phase and using the N-intein as a tag, but the former is preferred.

In one embodiment the C-intein and an additional tag is co-expressed with the POI. The additional tag may be any conventional chromatography tag, such as an IEX tag or an affinity tag.

### Methods of Purifying a Protein of Interest (POI)

The invention relates to a method for purification of a protein of interest (POI), using the split intein system according to the invention, comprising association of the C-intein and N-intein at neutral pH, such as 6-8, and in the presence of divalent cations (which impairs spontaneous cleavage); washing said solid phase in the presence of divalent cations; addition of a chelator to allow spontaneous cleavage between C-intein and POI; collection of tagless POI.

This protocol is suitable for protein non-sensitive for Zn. The advantages are long contact times are allowed with the resin and addition of large sample volume. Sample loading could be made for long times, such as up to 1.5 hours.

According to the invention more than 30% yield, preferably 50%, most preferably more than 80% of POI is achieved in less than 4 hours cleavage.

The invention enables a high ligand density when the N-intein is immobilized to a solid phase. Preferably the N-intein is attached to a chromatography resin, such as agarose or any other suitable resin for protein purification. According to the invention it is possible to achieve a static binding capacity of 0.2-2 μmole/ml C-intein bound POI per settled ml resin.

### Affinity Tags

The invention also relates to a method for purification of a protein of interest (POI), comprising the following steps: co-expressing a POI with a C-intein according to the invention and an additional tag; binding said additional tag to its binding partner on a solid phase; cleaving off the POI and the C-intein; binding said C-intein to an N-intein attached to a solid phase at neutral pH and cleaving off said bound C-intein and N-intein from said POI; and re-generating said solid phase under alkaline conditions, such as 0.5M NaOH. The purpose of this twin tag: increased purity (enables dual affinity purification), solubility, detectability.

Affinity tags can be peptide or protein sequences cloned in frame with protein coding sequences that change the protein's behavior. Affinity tags can be appended to the N- or C-terminus of proteins which can be used in methods of purifying a protein from cells. Cells expressing a peptide comprising an affinity tag can be expressed with a signal sequence in the supernatant/cell culture medium. Cells expressing a peptide comprising an affinity tag can also be pelleted, lysed, and the cell lysate applied to a column, resin or other solid support that displays a ligand to the affinity tags. The affinity tag and any fused peptides are bound to the solid support, which can also be washed several times with buffer to eliminate unbound (contaminant) proteins. A protein of interest, if attached to an affinity tag, can be eluted from the solid support via a buffer that causes the affinity tag to dissociate from the ligand resulting in a purified protein, or can be cleaved from the bound affinity tag using a soluble protease. As disclosed herein, the affinity tag is cleaved through the self-cleaving mechanism of the C-intein segment in the active intein complex.

Examples of affinity include, but are not limited to, maltose binding protein, which can bind to immobilized maltose to facilitate purification of the fused target protein; Chitin binding protein, which can bind to immobilized chitin; Glutathione S transferase, which can bind to immobilized glutathione; poly-histidine, which can bind to immobilized chelated metals; FLAG octapeptide, which can bind to immobilized anti-FLAG antibodies.

Affinity tags can also be used to facilitate the purification of a protein of interest using the disclosed modified peptides through a variety of methods, including, but not limited to, selective precipitation, ion exchange chromatography, binding to precipitation-capable ligands, dialysis (by changing the size and/or charge of the target protein) and other highly selective separation methods.

In some aspects, affinity tags can be used that do not actually bind to a ligand, but instead either selectively precipitate or act as ligands for immobilized corresponding binding domains. In these instances, the tags are more generally referred to as purification tags. For example, the ELP tag selectively precipitates under specific salt and temperature conditions, allowing fused peptides to be purified by centrifugation. Another example is the antibody Fc domain, which serves as a ligand for immobilized protein A or Protein G-binding domains.

### Proteins of Interest

Target proteins for all protocols are: any recombinant proteins, especially proteins requiring native or near native N-terminal sequences, for example therapeutic protein candidates, biologics, antibody fragments, antibody mimetics, protein scaffolds, enzymes, recombinant proteins or peptides, such as growth factors, cytokines, chemokines, hormones, antigen (viral, bacterial, yeast, mammalian) production, vaccine production, cell surface receptors, fusion proteins.

The invention will now be described more closely in association with some non-limiting examples and the accompanying drawings.

### Experimental Part

The invention will be described more closely in association with some non-limiting examples and the accompanying drawings.

In the present invention the following 5 constructs were evaluated:

TABLE 1

| | |
|---|---|
| A52 | ALSYETEILTVEYGLLPIGKIVEKRIECTVYSVDNNGNIYTQPVAQWHDRGEQEVFEYCL |
| A53 | ALSYDTEILTVEYGFLPIGKIVEENIECTVYSVDKNGFVYTQPIAQWHNRGEQEVFEYDL |
| B97_K24E/R25N | ALSYETEILTVEYGLLPIGKIVEENIECTVYSVDNNGNIYTQPVAQWHDRGEQEVFEYCL |
| B82_K24E | ALSYETEILTVEYGLLPIGKIVEERIECTVYSVDNNGNIYTQPVAQWHDRGEQEVFEYCL |
| B83_R25N | ALSYETEILTVEYGLLPIGKIVEKNIECTVYSVDNNGNIYTQPVAQWHDRGEQEVFEYCL |
| | ****:*********:********:.*********:** :****:****:********* * |
| A52 | EDGSLIRATKDHKFMTVDGQMLPIDEIFERELDLMRVGGSGDYKDDDDKGGSGHHHHHH |
| A53 | EDGSIIRATKDHKFMTTDGEMLPIDEIFEQGLDLKQVGGSGDYKDDDDKGGSGHHHHHH |
| B97_K24E/R25N | EDGSLIRATKDHKFMTVDGQMLPIDEIFERELDLMRVGGSGDYKDDDDKGGSGHHHHHH |
| B82_K24E | EDGSLIRATKDHKFMTVDGQMLPIDEIFERELDLMRVGGSGDYKDDDDKGGSGHHHHHH |
| B83_R25N | EDGSLIRATKDHKFMTVDGQMLPIDEIFERELDLMRVGGSGDYKDDDDKGGSGHHHHHH |
| | ****:***********.**:*********: *** :*********************** |
| | Non-Intein sequences |

### Construct Cultivation and Expression:

Start cultures were diluted 1:100 in 100 mL LB+neo in shake flasks (done in triplicates for all 5 constructs) and incubated at 37 until OD600 was 0.6-1. Once target OD was reached, the flasks were transferred to a cooled incubator (22 degrees) and 0.5 mM IPTG was added for induction over night (exactly 18 h). Induced cultivations were pelleted sequentially at 5000 g (+7 degrees) in 50 mL falcon tubes and pellets were weighed.

### Solubility Testing

Weighed pellets (ca 0.8 grams) were resuspended in 20 volumes of 1× PBS. For each of the constructs;
1. 20 μL was saved for SDS-PAGE (Whole cell Lysate/WCL)
2. 5 mL was added to 50 mL falcon tube and
   a. centrifuged for 20 min at 5000 g (6 degrees)
   b. pellet was resuspeded in 5 mL 8M Urea
   c. incubated by end-to-end for ˜1 h at room temp
   d. Centrifuged at 20000 g for 20 min at 6 degrees
   e. 20 μL of supernatant was saved for SDS-PAGE (Urea)
   f. Rest of supernatant was used for Biacore CFCA analysis
3. 10 mL was added to to a 50 mL falcon tube and
   a. Sonicated on ice for 1 min on time (2 sec on/4 sec off pulses) at 30% amplitude
   b. Centrifuged at 20000 g for 20 min at 6 degrees
   c. 20 μL of supernatant was saved for SDS-PAGE (Sonic.)
   d. Rest of supernatant was used for Biacore CFCA analysis
4. 500 μL was added to a 1.5 mL tube and
   a. Centrifuged for 20 min at 5000 g at 6 degrees
   b. Resuspended pellet in 500 μL 1% NP40 buffer
   c. Incubated for 40 min with 1200 rpm skaking (Mathilda block) at room temperature
   d. Centrifuged for 20 min at 12000 g at 6 degrees
   e. Saved ca 250 μL of the supernatant
   f. 20 μL of supernatant was saved for SDS-PAGE (NP40)
   g. Rest of supernatant was used for BiaCore CFCA analysis

To the 20 μL samples 40 μL SDS-PAGE buffer was added and boiled at 95 degrees for 5 min prior to loading the samples to a 15% gel.

### Solubility was Evaluated by SDS-PAGE Analysis Samples and Calculated Accordingly:

Extraction method densitometric signal/Whole cell lysate (WCL) densitometric signal*100%

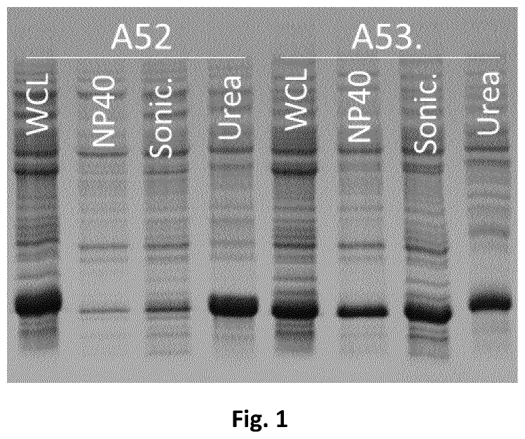

FIG. 1 shows SDS-PAGE analysis of representative supernatants after using different extraction techniques. 20 μL of supernatants were mixed with 40 μL of 2× Laemmli sample buffer and boiled for 5 minutes at 95 degrees celsius prior to loading on a 15% homogenous SDS-PAGE gel. Gel was electrophoresed for 1 h and 50 min at 600V and stained by coomassie for approximately two hours. After extensive destaining, gel was imaged using an Amersham AI600 imager.

FIG. 2 Shows solubility determined after densitometric evaluation of SDS-PAGE analysis. Extracts from three different cell-cultures for each construct were analysed and ligand band densitometry was measured using ImageQuant TL software. Solubility was calculated based on the following formula:

Densitometry(extraction method X)/Densitometry(WCL)*100%=% solubility

Bars show the average solubility of different extraction methods compared to whole cell lysate and the error bars show the standard deviation. All constructs showed significantly better solubility than A52 for the sonicated samples analysed. A53, B97 and B82 showed significantly higher solubility as compared to A52 samples. Statistical significance was determined by one-way ANOVA with Dunnet's post test using A52 as control sample. *p-value <0.05, ** p-value <0.01, *** p-value <0.001, **** p-value <0.0001.

| Construct name | NP40, mean solubility | Sonication, mean solubility |
|---|---|---|
| A52 | 7.4% | 22.1% |
| A53 | 67.6% | 104.7% |
| B97 | 37.3% | 92.8% |
| B82 | 44.3% | 62.8% |
| B83 | 12.4% | 36.6% |

Urea extraction method showed no significant difference as compared to WCL and independent of the construct analysed.

### Biacore CFCA Analysis

Determination of the soluble N-intein ratio of the various protein extracts was further analyzed by SPR binding analysis using a FLAG-epitope (DYKDDDDK) as a detection-tag at the C-terminus of the constructs. Calibration-free concentration analysis, (CFCA), was done in a Biacore T200 instrument using a mouse monoclonal ANTI-FLAG M2 antibody. Sensor chips, CM5 series S were immobilized with the anti-FLAG antibody using an amine coupling kit. 10 mM sodium acetate pH 4.0 was used as immobilization buffer, HBS-EP+pH 7.4 as a running buffer and Glycine-HCl pH 2.5 as regeneration buffer. The immobilization levels were about 8000-10000 RU. Supernatant samples from the different extractions described above, were diluted from 150-5000 times in HBS-EP+running buffer before anlysis by the CFCA method. The molecular weights of the different protein constructs ranged from 13.5-13.6 kDa and this was used to calculate the diffusion coefficient at 20° C., (1.13389E⁻¹⁰ m²/s). The default Biacore method for CFCA was used as a starting point for setting up the final method. Sample concentrations were determined by using the Biacore T200 evaluation software.

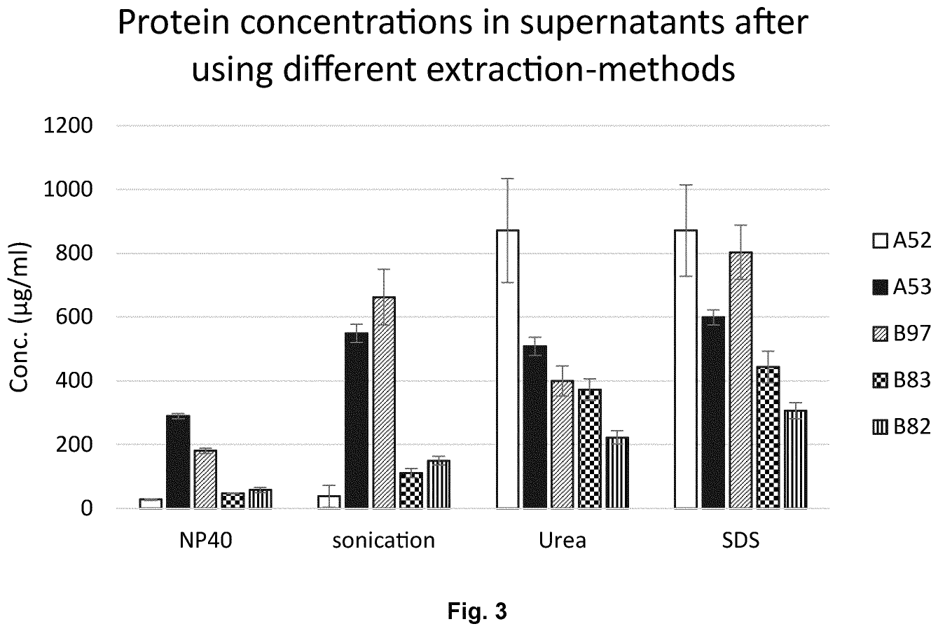

FIG. 3 shows N-intein concentrations in supernatants from different extracts determined by Biacore CFCA analysis. Extracts from three different cell-cultures for each construct were analysed. Bars show the average concentration and the error bars show the standard deviation.

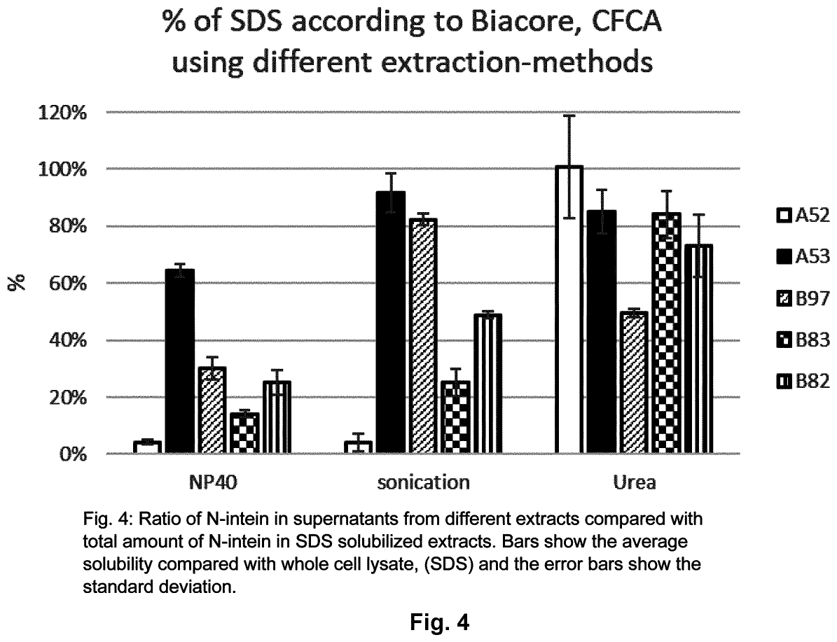

N-intein concentration in the supernatants after extraction and clarification using different extraction methods is used for the calculation of soluble N-intein ratios. The NP40 detergent buffer causes a mild release of soluble proteins from the cells. Ultra-sonication is a mechanical extraction technique causing vigorous cell disruption, releasing soluble proteins. Urea at high concentration is a denaturing extraction method causing the release of both soluble proteins and insoluble proteins found in inclusion bodies from the cells. Boiling of the cell pellets in a SDS sample buffer causes complete solubilization of both soluble and insoluble N-intein and is used as the reference for total amount of expressed N-intein. A high N-intein concentration in supernatants after extraction with non-denaturing extraction methods, (NP40 and sonication) compared with denaturing methods, (Urea and SDS) indicate a high solubility. The CFCA analysis show that the A53 and B97 constructs have a high solubility whereas A52 has a very poor solubility, FIG. 4.

Statistical analysis show that the modified constructs B82, B83 and B97 are significantly more soluble compared with the non-modified A52 construct when using mild non-denaturing extraction methods.

### Solubility Evaluated by SPR Binding Analysis is Calculated Accordingly:

Extraction method N-intein concentration/SDS extracted N-intein concentration*100%

### Results

Sodium dodecyl sulfate, SDS, is an ionic detergent that binds to proteins through ionic and hydrophobic interactions and solubilizes proteins by altering their secondary and tertiary structure. SDS is routinely used in polyacrylamide gel electrophoresis, (SDS-PAGE) to separate, characterize and quantify proteins. SDS has been used in these example experiments as a universal protein solubilizing reagent used for quantification of the total amount of protein in different extracts, both soluble and insoluble for subsequent separation, detection and quantification by densitometric analys of SDS-PAGE gels and Biacore calibration free concentration analysis, CFCA. The concentration of different constructs in SDS solubilized sample extracts is normalized to 100% for comparison with the concentration of the different protein constructs derived in the supernatants after centrifugal clarification of extracts using different methods.

A mild method for extracting soluble proteins only is the use of a non-ionic detergent NP40. NP40 at 1% (w/v) is added to a Tris-HCl buffer, pH 7.5 containing 150 mM sodium chloride and is simply used by resuspending harvested bacterial cell pellets followed by mixing during 1 hour. After incubation the cell suspension is clarified by centrifugation to remove insoluble material.

Ultra-sonication or sonication, is an extraction method for proteins that uses mechanical energy from a probe to disintegrate cells for the release of soluble cell components. Cells are resuspended in a non-denaturing buffer like phosphate buffered saline, PBS at pH 7.4 to control the pH during the release of cellular components. Sonication is a very efficient and reliable tool for cell disintegration that allows for a complete control over the sonication parameters. This ensures a high selectivity on materials release and product purity. After sonication, the lysate is clarified by supernatant and the insoluble pellet is removed.

Chaotropic salts like Urea can be used for the release of both soluble and insoluble proteins from cells. Urea is compatible with a wide range range of analytical methods in contrast to SDS detergent that is more likely to interfere with some commonly used analytical methods. Urea is commonly used at 8 M to ensure maximum denaturing conditions and can be dissolved in water. Cells are resuspended in the Urea solution followed by mixing during 1 hour. The extract is then clarified by centrifugation to remove the insoluble pellet.

SDS denatures proteins when heated and imparts a strong negative charge to all proteins. SDS binds strongly to proteins in the ratio of one SDS molecule per two amino acids. This makes SDS extraction a very efficient method to assess the amount of total protein, both soluble and insoluble. In general, a 2% (w/v) SDS concentration in a buffer solution between pH 6.7-7.5 is added to an equal volume of cell suspension from a cell harvest followed by mixing and heating at 95° C. for 5 minutes. Then the samples are cooled down to room temperature before centrifugation and analysis.

FIG. 3. shows the concentration of different N-intein constructs in the supernatants after extraction of proteins in the cell harvest by the use of different methods. The amount of cells and the extraction volumes were normalized prior to extraction so that the actual concentration can be directly compared. Each bar shows the average concentration for a certain construct derived from the extraction of cells from three different cell cultures. The error bars show the standard deviation. As can be seen in FIG. 3. the concentration of the protein constructs are highest in the supernatants after extraction using SDS and Urea. The relative difference between the concentration of the different constructs reflect a varying degree of expression from the different cell cultures. Constructs A52 and B97 had the highest N-intein expression in total according to concentration in the SDS extracts, 871 and 803 μg/ml respectively. N-intein concentrations from the Urea extracts are generally lower compared with SDS extracts but follows roughly the same pattern. The interesting findings can be seen in the N-intein concentrations from the sonication and NP40 extracts where only the soluble proteins are found. A52, a construct that does not comprise the substitution mutations K24E or R25N has the lowest concentration of N-intein compared with the other constructs with 27.5 μg/ml in NP40 extracts and 37.9 μg/ml in sonicated samples. The construct B97 comprising the K24E and R25N substitutions has a relatively high concentration of soluble N-intein in NP40 extracts, 180.3 μg/ml and in sonicated extracts, 662.3 μg/ml. This difference is more pronunced in FIG. 4., where the N-intein concentration for each respective construct and extraction method is compared with the N-intein concentration after SDS extraction of each respective construct. SDS bars are omitted since they all give the ratio 1, equal to 100%. The construct A52 lacking the mutations at position 24 and 25 has only 3 and 4% N-intein in extracts from NP40 and sonication respectively compared with SDS extracts. A single substitution, R25N in construct B83 results in a higher ratio relative to SDS extracts, 11% and 25% respectively for NP40 and sonication extracts. A single substitution, K24E in construct B82 results in a higher ratio relative to SDS extracts, 19% and 49% respectively for NP40 and sonication extracts. Construct B97, with two amino acid substitutions at position 24 and 25, K24E and R25N, results in a higher ratio relative to SDS extracts, 22% and 82% respectively for NP40 and sonication extracts.

In summary, the solubility ranges achieved according to the invention in the above experiments are:

At least 10-40% soluble N-intein with a single-point mutation of R at position 25, preferred N or non-positive amino acid. At least 46-52% soluble N-intein with a single-point mutation of K at position 24, preferred E or non-positive amino acid. At least 76-88% soluble N-intein with mutations at positions 24 and 25, preferred K24E and R25N or non-positive amino acids. These values are based on Biacore CFCA data on sonicated samples.

1. An N-intein protein variant derived from wildtype Nostoc punctiforme (Npu) or sequences having at least 95% homology therewith comprising at least one amino acid substitution of a native split intein, wherein the N-intein protein variant sequence includes a mutation in at least position 24 and/or position 25 as measured from the initial catalytic cysteine and wherein the substituted amino acid provides increased solubility in aqueous buffers compared to the native N-intein protein sequence or a consensus N-intein sequence.

2. The N-intein protein variant of claim 1 wherein the substituted amino acid(s) that provide increased solubility is a non-positive amino acid.

3. The N-intein protein variant of claim 1, wherein the substituted amino acid that provide increased solubility is K24E.

4. The N-intein protein variant of claim 1, wherein the substituted amino acid that provide increased solubility is R25N.

5. An N-intein protein variant of the wildtype N-intein domain of Nostoc punctiforme (Npu) wherein the wildtype Npu N-intein domain comprises the following sequence:
CLSYETEILTVEYGLLPIGKIVEKRIECTVYSVDNNGNIYTQPVAQWHDRGEQEVFE YCLEDGSLIRATKDHKFMTVDGQMLPIDEIFERELDLMRV (SEQ ID NO 1), wherein the protein variant comprises an amino acid substitution from K to E in position 24 of SEQ ID NO 1 and R to N in position 25 of SEQ ID NO 1 to increase solubility in aqueous buffers, and wherein optionally one or more C is/are mutated to non-Cystein residues, preferably S or A.

6. The N-intein protein variant of claim 1, wherein the solubility in aqueous buffer is at least 10-40% soluble N-intein with a single-point mutation of R at position 25, preferred N or non-positive amino acid; at least 46-52% soluble N-intein with a single-point mutation of K at position 24, preferred E or non-positive amino acid; and at least 76-88% soluble N-intein with mutations at positions 24 and 25, preferred K24E and R25N or non-positive amino acids.

7. The N-intein protein variant according to claim 1, which is attached to a solid phase, such as a membrane, fiber, particle, bead or chip.

8. The N-intein protein variant sequence according to claim 7, wherein the solid phased is a chromatography resin of natural or synthetic origin.

9. The N-intein protein variant according to claim 7, wherein the solid phase is a chromatography resin, such as a natural or synthetic resin, preferably a polysaccharide such as agarose.

10. The N-intein protein variant according to claim 9, wherein the solid phase is provided with embedded magnetic particles.

11. The N-intein protein variant according to claim 9, wherein the solid phase is a non-diffusion limited resin/fibrous material.

12. The N-intein protein variant according to claim 1, wherein the N-intein is coupled to the solid phase via a Lys-tail, comprising one or more Lys, on the C-terminal.

13. The N-intein protein variant according to claim 1, wherein the N-intein is coupled to the solid phase via a Cys-tail on the C-terminal.

14. The N-intein protein variant according to claim 1, wherein 0.2-2 μmole/ml N-intein is coupled per ml solid phase, preferably chromatography resin (ml swollen gel).

15. A split intein system comprising a N-intein protein variant according to claim 1, attached to a solid phase, and a C-intein sequence which is co-expressed with a POI (protein of interest), wherein the C-intein acts as a tag on the POI and the expressed C-intein binds to said N-intein protein variant.

16. Split intein system according to claim 15, wherein the C-intein sequence is a native split intein C-intein sequence or engineered variants thereof.

17. Split intein system according to claim 15, wherein the POI's are: proteins requiring native or near native N-terminal sequences, for example therapeutic protein candidates, biologics, antibody fragments, antibody mimetics, enzymes, recombinant proteins or peptides, such as growth factors, cytokines, chemokines, hormones, antigen (viral, bacterial, yeast, mammalian) production, vaccine production, cell surface receptors, fusion proteins.


SEQUENCE LISTING (1 sequence)

SEQ ID NO: 1 - protein, 97 aa, Nostoc punctiforme
CLSYETEILTVEYGLLPIGKIVEKRIECTVYSVDNNGNIYTQPVAQWHDRGEQEVFEYCLEDGSLIRATKDHKFMTVDGQMLPIDEIFERELDLMRV